(19) United States
(12) Patent Application Publication
(10) Pub. No.: US 2023/0021647 A1
(43) Pub. Date: Jan. 26, 2023

(54) METHOD OF TREATING A PATIENT INFECTED WITH A CORONAVIRUS AND HAVING A BASELINE LEVEL OF CRP BELOW 150 MG/L

(71) Applicant: Theravance Biopharma R&D IP, LLC, South San Francisco, CA (US)

(72) Inventors: Rajeev Saggar, Scottsdale, AZ (US); Tuan Stevon Nguyen, Pleasanton, CA (US); David A. Lombardi, Natick, MA (US); Glenn D. Crater, Raleigh, NC (US); Edmund J. Moran, San Francisco, CA (US); Nathan David Pfeifer, Montara, CA (US); Wayne Arthur Yates, San Mateo, CA (US); Kyla Dee-Kinnick Kennedy, Clive, IA (US); Joseph Budman, Redwood City, CA (US); Arthur Lo, San Francisco, CA (US)

(21) Appl. No.: 17/844,593

(22) Filed: Jun. 20, 2022

Related U.S. Application Data
(60) Provisional application No. 63/202,698, filed on Jun. 21, 2021.

(51) Int. Cl. A61K 31/437; A61K 31/573; A61K 31/675; A61P 31/14

(52) U.S. Cl. CPC A61K 31/437; A61K 31/573; A61K 31/675; A61P 31/14

(57) ABSTRACT

Provided herein are methods of treating a patient infected with a coronavirus and having a baseline level of CRP below 150 mg/L, comprising administering to the patient a compound of formula 1:

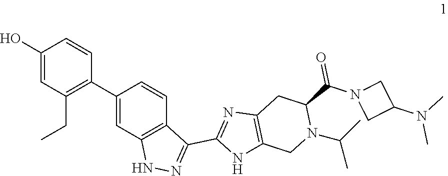 [SMILES: CCc1cc(O)ccc1-c1ccc2c(-c3nc4c([nH]3)CN(C(C)C)[C@H](C(=O)N3CC(N(C)C)C3)C4)n[nH]c2c1]

or a pharmaceutically-acceptable salt thereof.

### CROSS-REFERENCE TO RELATED APPLICATIONS

This application claims priority to U.S. Provisional Application No. 63/202,698, filed Jun. 21, 2021, which is hereby incorporated by reference in its entirety.

### BACKGROUND

### Field

Provided herein is a method of treating a patient infected with a coronavirus having a baseline level of CRP (C-reactive protein) below 150 mg/L.

### State of the Art

Human coronavirus is a common respiratory pathogen and typically induces mild upper respiratory disease. The two highly pathogenic viruses, Severe Acute Respiratory Syndrome-associated Coronavirus (SARS-CoV-1) and Middle East Respiratory Syndrome-associated Coronavirus (MERS-CoV), caused severe respiratory syndromes resulting in more than 10% and 35% mortality, respectively (Assiri et al., N Engl J Med., 2013, 369, 407-1). The recent emergence of Coronavirus Disease 2019 (COVID-19) and the resulting pandemic has created a global health care emergency. Similar to SARS-CoV-1 and MERS-CoV, a subset of patients (about 16%) can develop a severe respiratory illness manifested by acute lung injury (ALI) leading to ICU admission (about 5%), respiratory failure (about 6.1%) and death (Wang et al., JAMA, 2020, 323, 11, 1061-1069; Guan et al., N Engl J Med., 2020, 382, 1708-1720; Huang et al., The Lancet, 2020. 395 (10223), 497-506; Chen et al., The Lancet, 2020, 395(10223), 507-13). Accumulating evidence suggests that a subgroup of patients with COVID-19 might have a hyperinflammatory “cytokine storm” resulting in acute lung injury and acute respiratory distress syndrome (ARDS). This cytokine storm may also spill over into the systemic circulation and produce sepsis and ultimately, multi-organ dysfunction syndrome (Zhou et al., The Lancet, 2020, Vol. 395, Issue 10229, 1054-1062). The dysregulated cytokine signaling that appears in COVID-19 is characterized by increased expression of interferons (IFNs), interleukins (ILs), and chemokines, resulting in ALI and associated mortality.

Concerns have been raised about the potential increased risk for thromboembolism with systemic JAK inhibitors, which is particularly concerning given observations of severe hypercoagulability in patients with COVID-19. A lung-selective, inhaled pan-JAK inhibitor would address the shortcomings of oral JAK inhibitors by avoiding systemic immunosuppression, thromboembolisms, and additional infections that may lead to worsened mortality. As major causes of death in subjects with COVID-19 appear to be comorbidities, thromboembolism, and superinfection, an inhaled medication may be a way to avoid systemic immunosuppression that would pre-dispose patients to these risks.

There is a need for safe and effective treatments, including for subpopulations of patients, that can be used alone or in combination with standard of care that can dampen the cytokine storm associated with a coronavirus infection.

### SUMMARY

Provided herein are methods of treating a patient infected with a coronavirus and having a baseline level of CRP below 150 mg/L, comprising administering to the patient a compound of formula 1:

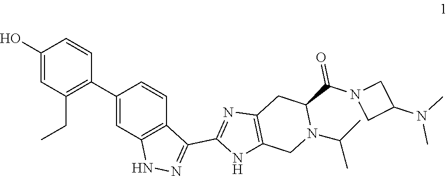 [SMILES: CCc1cc(O)ccc1-c1ccc2c(-c3nc4c([nH]3)CN(C(C)C)[C@H](C(=O)N3CC(N(C)C)C3)C4)n[nH]c2c1]

or a pharmaceutically-acceptable salt thereof.

### DETAILED DESCRIPTION

The compound of formula 1 (also referred to herein as “compound 1”) is named (S)-(3-(dimethylamino)azetidin-1-yl)(2-(6-(2-ethyl-4-hydroxyphenyl)-1H-indazol-3-yl)-5-isopropyl-4,5,6,7-tetrahydro-3H-imidazo[4,5-c]pyridin-6-yl)methanone) and has the structure:

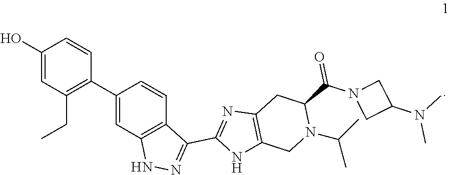 [SMILES: CCc1cc(O)ccc1-c1ccc2c(-c3nc4c([nH]3)CN(C(C)C)[C@H](C(=O)N3CC(N(C)C)C3)C4)n[nH]c2c1]

The compound of formula 1 is a pan-JAK inhibitor suitable for direct delivery to the lungs which was first disclosed in U.S. application Ser. No. 16/559,077 (US Pat. Pub. 2020/0071323), filed on Sep. 3, 2019.

Provided herein are methods of treating a patient infected with a coronavirus and having a baseline level of CRP below 150 mg/L, comprising administering to the patient a compound of formula 1:

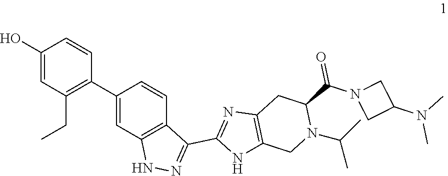 [SMILES: CCc1cc(O)ccc1-c1ccc2c(-c3nc4c([nH]3)CN(C(C)C)[C@H](C(=O)N3CC(N(C)C)C3)C4)n[nH]c2c1]

or a pharmaceutically-acceptable salt thereof.

In some embodiments, the coronavirus is selected from the group consisting of SARS-CoV-1, SARS-CoV-2, and MERS-CoV. In some embodiments, the coronavirus is SARS-CoV-2.

In some embodiments, the coronavirus viral load is reduced in the respiratory system. In some embodiments, the coronavirus viral load is reduced in the lungs. In some embodiments, the reduction in coronavirus viral load is measured by collecting and analyzing nasal swabs from the patient. In some embodiments, viral load of a patient prior to administration of compound 1, or a pharmaceutically-acceptable salt thereof, is compared to viral load of the patient after such administration to determine if there is a reduction in viral load. In some embodiments, the method reduces the viral load of SARS-CoV-2 in the respiratory system of the patient. In some embodiments, the method reduces the viral load of SARS-CoV-2 in the lungs of the patient. In some embodiments, the reduction in viral load is measured by collecting and analyzing nasal swabs from the patient.

In some embodiments, the compound of formula 1, or a pharmaceutically-acceptable salt thereof, is administered by inhalation. In some embodiments, the compound of formula 1, or a pharmaceutically-acceptable salt thereof, is administered as a dry-powder composition. In some embodiments, the compound of formula 1, or a pharmaceutically-acceptable salt thereof, is administered with a dry powder inhaler. In some embodiments, the compound of formula 1, or a pharmaceutically-acceptable salt thereof, is administered by nebulized inhalation.

In some embodiments, the compound, or a pharmaceutically-acceptable salt thereof, is administered to the patient in an outpatient setting. In some embodiments, the compound, or a pharmaceutically-acceptable salt thereof, is administered to the patient wherein the patient is not hospitalized. In some embodiments, the compound, or a pharmaceutically-acceptable salt thereof, is administered to the patient before hospitalization. In some embodiments, the compound, or a pharmaceutically-acceptable salt thereof, is administered to the patient during hospitalization. In some embodiments, hospitalization refers to admittance of a patient to a hospital or a patient staying at a hospital for at least 24 hours.

In some embodiments, the patient suffers from one or more of hypoxia, hypoxemia, dyspnea, shortness of breath, and low oxygen levels. In some embodiments, the patient requires supplemental oxygen. In some embodiments, the patient requires supplemental oxygen to maintain an oxygen saturation over 90%. In some embodiments, the patient is under oxygen, non-invasive ventilation, or mechanical ventilation.

In some embodiments, the compound, or a pharmaceutically-acceptable salt thereof, is administered once a day. In some embodiments, the compound, or a pharmaceutically-acceptable salt thereof, is administered twice a day.

In some embodiments, the compound, or a pharmaceutically-acceptable salt thereof, is administered at a higher loading dose on day 1 of administration followed by a lower dose on the following days.

In some embodiments, the compound, or a pharmaceutically-acceptable salt thereof, is administered at a dose of 0.1 mg to 100 mg per day. In some embodiments, the compound, or a pharmaceutically-acceptable salt thereof, is administered at a dose of 1 mg to 20 mg per day.

In some embodiments, the compound of formula 1, or a pharmaceutically-acceptable salt thereof, is administered to the patient at a dose of about 3 mg to about 10 mg. In some embodiments, the compound of formula 1, or a pharmaceutically-acceptable salt thereof, is administered to the patient at a dose of about 1 mg. In some embodiments, the compound of formula 1, or a pharmaceutically-acceptable salt thereof, is administered to the patient at a dose of about 3 mg. In some embodiments, the compound of formula 1, or a pharmaceutically-acceptable salt thereof, is administered to the patient at a dose of about 10 mg. In some embodiments, the compound of formula 1, or a pharmaceutically-acceptable salt thereof, is administered to the patient at a dose of about 3 mg daily. In some embodiments, the compound of formula 1, or a pharmaceutically-acceptable salt thereof, is administered to the patient at a single daily dose of about 3 mg. In some embodiments, the compound of formula 1, or a pharmaceutically-acceptable salt thereof, is administered to the patient at a single daily dose of about 3 mg with a loading dose of about 6 mg on the first day of administration. In some embodiments, the compound of formula 1, or a pharmaceutically-acceptable salt thereof, is administered to the patient at a single daily dose of about 1 mg with a loading dose of about 2 mg on the first day of administration.

In some embodiments, compound 1, or a pharmaceutically acceptable salt thereof, is administered at a daily dose of about 0.5 mg, 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 20 mg, 25 mg, 30 mg, 35 mg, 40 mg, 45 mg, 50 mg, 75 mg, or 100 mg.

In some embodiments, the compound, or a pharmaceutically-acceptable salt thereof, is administered for 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 2 weeks, 3 weeks, 4 weeks, 1 month, 5 weeks, 6 weeks, 2 months, or 3 months. In some embodiments, the compound, or a pharmaceutically-acceptable salt thereof, is administered until discharge of the patient from the hospital. In some embodiments, the compound of formula 1, or a pharmaceutically-acceptable salt thereof, is administered to the patient for up to 7 days, or up to 10 days, or up to 14 days, or until discharge from the hospital. In some embodiments, the compound of formula 1, or a pharmaceutically-acceptable salt thereof, is administered to the patient for up to 7 days, or until discharge from the hospital. In some embodiments, the compound of formula 1, or a pharmaceutically-acceptable salt thereof, is administered to the patient for up to 14 days, or until discharge from the hospital.

In some embodiments, the method comprises administering one or more additional therapeutic agents or treatments to the patient.

In some embodiments, the methods described herein may achieve certain clinical improvements (e.g. decreases/reductions or increases/additions as described herein) in a patient's condition compared to the patient's condition to prior to administration of the compound of formula 1.

In some embodiments, the method decreases inflammation in the lungs caused by the coronavirus.

In some embodiments, the method prevents, reduces, or resolves acute lung injury and/or acute respiratory distress syndrome caused by the coronavirus. In some embodiments, the method prevents, reduces, or stops a cytokine storm caused by the coronavirus.

In some embodiments, the method results in an increase in oxygen levels in the blood of the patient. In some embodiments, the method results in an improvement or resolution of fever in the patient. In some embodiments, the method results in removal of the patient from ventilation or oxygen supplementation. In some embodiments, the method increases the number of ventilator free days in the patient. “Ventilator free day (VFD)” as used herein refers to a day that a subject is alive and successfully not using invasive mechanical ventilation or non-invasive positive pressure ventilation. In some embodiments, the method increases ICU (Intensive Care Unit) free days for the patient. “ICU free day” as used herein refers to a day that a subject does not require care in an Intensive Care Unit. In some embodiments, the method results in an improvement or resolution of shortness of breath. In some embodiments, the method results in a lower risk of mortality in the patient.

In some embodiments, the method reduces the viral load of the coronavirus in the respiratory system of the patient. In some embodiments, the method reduces the viral load of the coronavirus in the lungs of the patient. In some embodiments, the reduction in viral load is measured by collecting and analyzing nasal swabs from the patient.

In some embodiments, the compound, or a pharmaceutically-acceptable salt thereof, is administered by inhalation. In some embodiments, the compound, or a pharmaceutically-acceptable salt thereof, is administered by nebulized inhalation. In some embodiments, the compound, or a pharmaceutically-acceptable salt thereof, is administered as a dry-powder composition. In some embodiments, the compound, or a pharmaceutically-acceptable salt thereof, is administered with a dry powder inhaler.

In some embodiments, the method decreases inflammation in the lungs caused by COVID-19. In some embodiments, the method prevents, reduces, or resolves acute lung injury and/or acute respiratory distress syndrome caused by COVID-19. In some embodiments, the method prevents, reduces, or stops a cytokine storm caused by COVID-19. In some embodiments, the method results in an increase in oxygen levels in the blood of the patient. In some embodiments, the method results in an improvement or resolution of fever in the patient.

In some embodiments, the method results in removal of the patient from ventilation or oxygen supplementation. In some embodiments, the method increases the number of ventilator free days in the patient. In some embodiments, the method increases ICU (Intensive Care Unit) free days for the patient. In some embodiments, the method results in an improvement or resolution of shortness of breath. In some embodiments, the method results in a lower risk of mortality in the patient.

In some embodiments, the method prevents hospitalization of the patient. In some embodiments, the method prevents serious complications in the patient. In some embodiments, the serious complications include lung injury, acute lung injury (ALI), acute respiratory distress syndrome (ARDS), organ failure, pneumonia, acute liver injury, blood clots, respiratory failure, need for oxygen supplementation, non-invasive ventilation, or mechanical ventilation, acute cardiac injury, a secondary infection, acute kidney injury, septic shock, disseminated intravascular coagulation, multisystem inflammatory syndrome in children (MIS-C), rhabdomyolysis, arrhythmia, a cytokine storm, and cardiovascular shock.

In some embodiments the patient is at a high risk of developing severe complications from the coronavirus. In some embodiments, the patient has been identified as being at a high risk of developing severe complications from the coronavirus through biomarker testing. In some embodiments, the patient has been identified as being at a high risk of developing severe complications from the coronavirus based on LDH (lactate dehydrogenase) levels. In some embodiments, the patient has been identified as being at a high risk of developing severe complications from the coronavirus based on LDH-isoform3 levels. In some embodiments, the patient has been identified as being at a high risk of developing severe complications from the coronavirus based on levels of surfactant protein D (SP-D), receptor for advanced glycation end products (RAGE), one or more cytokines, C-reactive protein (CRP), D-dimer, fibrinogen, and/or ferritin. In some embodiments, the cytokine is IL-6. In some embodiments, the patient has diabetes, obesity, a cardiovascular disease (such as a coronary artery disease, myocardial infarction, a history of cerebrovascular accident, or peripheral arterial disease), hypertension, or a lung disease. In some embodiments, the patient has coronary artery disease, myocardial infarction, a history of cerebrovascular accident, peripheral arterial disease, or a lung disease (such as asthma, COPD, or IPF). In some embodiments, the patient has been identified as being at a high risk of developing severe complications from the coronavirus based on a chest x-ray. In some embodiments, the patient has a CXR abnormality consistent with viral pneumonia.

In some embodiments, the method decreases the rate of medical intervention associated with the coronavirus. In some embodiments, the rate of medical intervention associated with the coronavirus is measured by the number of emergency visits, hospitalization, physician visits, and urgent care visits associated with the coronavirus.

In some embodiments, the method decreases inflammation in the lungs caused by the coronavirus. In some embodiments, the method prevents, reduces, or resolves acute lung injury and/or acute respiratory distress syndrome caused by the coronavirus. In some embodiments, the method prevents, reduces, or stops a cytokine storm caused by the coronavirus. In some embodiments, the method results in an increase in oxygen levels in the blood of the patient. In some embodiments, the method results in an improvement or resolution of fever in the patient. In some embodiments, the method results in an improvement or resolution of shortness of breath. In some embodiments, the method results in a lower risk of mortality in the patient. In some embodiments, the method results in an improvement in the Patient Global Assessment of Symptoms of the patient. In some embodiments, the method results in an improvement in the Patient Global Rating of Change of the patient. In some embodiments, the method results in an improvement in levels of LDH (lactate dehydrogenase), surfactant protein D (SP-D), receptor for advanced glycation end products (RAGE), one or more cytokines, C-reactive protein (CRP), D-dimer, fibrinogen, and/or ferritin in the patient. In some embodiments, the cytokine is IL-6.

In some embodiments the compound inhibits viral entry or fusion of the coronavirus virions with the endosomal membrane in the cells of the patient. In some embodiments the compound inhibits Abelson kinases in the patient. In some embodiments, the Abelson kinases are Abl1 and Abl2. In some embodiments, the Abelson kinase is Abl1. In some embodiments, the Abelson kinase is Abl2. In some embodiments the compound inhibits replication of the coronavirus in the patient.

In some embodiments, the patient is in an outpatient setting.

In some embodiments, the method prevents serious complications in the patient. In some embodiments, the serious complications include lung injury, ALI, ARDS, organ failure, pneumonia, acute liver injury, blood clots, respiratory failure, need for oxygen supplementation, non-invasive ventilation, or mechanical ventilation, acute cardiac injury, a secondary infection, acute kidney injury, septic shock, disseminated intravascular coagulation, MISC, Rhabdomyolysis, arrhythmia, a cytokine storm, and cardiovascular shock.

In some embodiments, the method decreases the rate of medical intervention associated with the coronavirus. In some embodiments, the rate of medical intervention associated with the coronavirus is measured by the number of emergency visits, hospitalization, physician visits, and urgent care visits associated with the coronavirus.

In some embodiments, the patient has COVID-19-associated inflammatory syndrome (i.e. Multisystem Inflammatory Syndrome in children, MIS-C). In some embodiments, the patient is a pediatric patient.

In some embodiments, the method results in a decreased time to recovery and/or time to discharge from a hospital or medical facility.

In some embodiments, the method prevents long-term lung dysfunction or “long haul COVID” in the patient.

In some embodiments, the method prevents severe complications in the patient.

In some embodiments, the method results in an increase in the number of RFDs (Respiratory failure-free days) for a patient infected with a coronavirus.

In some embodiments, the method results in a decrease in hospitalization time and/or decreasing time in the ICU and/or decreasing time to discharge.

In some embodiments, the method increases the PaO₂/FiO₂ ratio (ratio of arterial oxygen partial pressure to fractional inspired oxygen) in the patient.

In some embodiments, the method decreases the mortality rate in the patient population.

In some embodiments, the method decreases blood clot formation in the patient.

In some embodiments, the method improves the Borg Dyspnea Score in the patient. In some embodiments, the method decreases C Reactive protein levels (CRP) in the patient. In some embodiments, the method decreases D-dimer levels in the patient. In some embodiments, the method decreases of cytokine levels in the patient. In some embodiments, the cytokine is IL-6.

In some embodiments, the method results in a decrease for the need for oxygen supplementation, non-invasive ventilation, or mechanical ventilation. in the patient.

In some embodiments, the patient is classified as moderate, severe, or critical. In some embodiments, the patient is 60 years old or less. In some embodiments, the patient is older than 60 years old. In some embodiments, the patient suffers from pneumonia when the compound, or a pharmaceutically acceptable salt thereof, is administered. In some embodiments, the patient suffers from bilateral pneumonia when the compound, or a pharmaceutically acceptable salt thereof, is administered. In some embodiments, the administration of the compound, or a pharmaceutically acceptable salt thereof, results in: prevention or attenuation of the formation of lung lesions, lung lesion opacity, lung injury, improvement in the patient monitored by chest imaging, CT scan or chest x-ray, increase in the number of ventilator-free days (VFDs), increase in the number of ICU-free days, increase in PaO₂/FiO₂ ratio, or increase in SaO₂/FiO₂ ratio (ratio of partial pressure of oxygen in arterial blood to the fraction of inspired oxygen). In some embodiments, administration of compound 1, or a pharmaceutically-acceptable salt thereof, to a coronavirus patient is done when the patient is in respiratory failure but before mechanical ventilation is needed.

In some embodiments, the method results in an increase in RFDs (Respiratory failure-free day) for the patient, the method results in a decrease in the need for supplemental oxygen for the patient, and/or the method results in an increase in days without supplemental oxygen for the patient.

In some embodiments, the compound, or a pharmaceutically-acceptable salt thereof, is administered to the patient at a key inflection point of the disease before ALI develops and prevents progression to ALI. In some embodiments, the compound, or a pharmaceutically-acceptable salt thereof, is administered to the patient during the early stages of the coronavirus infection, before ALI develops and the administration prevents progression to ALI. In some embodiments, the compound, or a pharmaceutically-acceptable salt thereof, is administered for a short period of time. In some embodiments, the compound, or a pharmaceutically-acceptable salt thereof, is administered to the patient before ARDS develops and prevents progression to ARDS.

In some embodiments, the compound, or a pharmaceutically-acceptable salt thereof, is administered to a patient that has not been admitted to a hospital, i.e. an outpatient. In some embodiments, the patient has one or more underlying conditions, such as asthma, COPD, a cardio-vascular disease, diabetes, chronic lung disease, a heart condition, cancer, a bone marrow or organ transplantation, an immune deficiency, HIV, takes an immune weakening medication, obesity, chronic kidney disease, a neurodevelopmental condition, high blood pressure, or liver disease.

In some embodiments, the patient is 80 years old or older, 70 years old or older, 65 years old or older, 60 years old or older, 50 years old or older, 40 years old or older, 10 years old or younger, between 10 and 20 years old, between 20 and 30 years old, between 30 and 40 years old, between 40 and 50 years old, between 50 and 60 years old, between 20 and 40 years old, between 40 and 60 years old, between 60 and 80 years old, 60 years old or younger, or over 60 years old. In some embodiments, the patient is 16 years old or older. In some embodiments, the patient is 18 years old or older. In some embodiments, the patient is 12 years old or older.

In some embodiments, administration of compound 1, or a pharmaceutically-acceptable salt thereof, to a coronavirus patient is done before the patient has ALI and/or ARDS, in order to prevent ALI and/or ARDS in the patient. In some embodiments, the coronavirus is selected from the group consisting of SARS-CoV-1, SARS-CoV-2, and MERS-CoV.

In some embodiments, the method blocks or inhibits neutrophilia and/or the formation of neutrophil extracellular traps (NETs) in the patient.

In some embodiments, the method decreases the risk of thrombosis or thromboembolism in the patient.

In some embodiments, the method decreases the incidence of thrombosis or thromboembolism in the patient population.

In some embodiments, the maximum plasma concentration (Cmax) in the patient of the compound of formula 1 is under 350 ng/mL. In some embodiments, the maximum plasma concentration in the patient of the compound of formula 1 is under 300 ng/mL. In some embodiments, the maximum plasma concentration in the patient of the compound of formula 1 is under 250 ng/mL. In some embodiments, the maximum plasma concentration in the patient of the compound of formula 1 is under 200 ng/mL. In some embodiments, the maximum plasma concentration in the patient of the compound of formula 1 is under 150 ng/mL. In some embodiments, the maximum plasma concentration in the patient of the compound of formula 1 is under 100 ng/mL. In some embodiments, the maximum plasma concentration in the patient of the compound of formula 1 is under 50 ng/mL. In some embodiments, the maximum plasma concentration in the patient of the compound of formula 1 is under 40, 30, 25, 20, 15, or 10 ng/mL. In some embodiments, the maximum plasma concentration in the patient of the compound of formula 1 is under the plasma concentration necessary to achieve JAK IC₅₀. In some embodiments, the JAK IC₅₀ is calculated by determining the IC₅₀ for IL-13-induced STAT6 phosphorylation in the human bronchial epithelial cell line BEAS-2B. In some embodiments, the maximum plasma concentration in the patient of the compound of formula 1 is under the plasma concentration necessary to inhibit Janus kinases by 50%.

In some embodiments, the compound of formula 1, or a pharmaceutically-acceptable salt thereof, is administered to the patient at a dose of about 1 mg to about 10 mg. In some embodiments, the compound of formula 1, or a pharmaceutically-acceptable salt thereof, is administered to the patient at a dose of about 1 mg. In some embodiments, the compound of formula 1, or a pharmaceutically-acceptable salt thereof, is administered to the patient at a dose of about 3 mg. In some embodiments, the compound of formula 1, or a pharmaceutically-acceptable salt thereof, is administered to the patient at a dose of about 10 mg. In some embodiments, the compound of formula 1, or a pharmaceutically-acceptable salt thereof, is administered to the patient at a dose of about 1 mg to about 3 mg. In some embodiments, the compound of formula 1, or a pharmaceutically-acceptable salt thereof, is administered to the patient at a dose of about 3 mg to about 10 mg. In some embodiments, the compound of formula 1, or a pharmaceutically-acceptable salt thereof, is administered to the patient at a dose of about 2 mg, or about 4 mg, or about 5 mg, or about 6 mg, or about 7 mg, or about 8 mg, or about 9 mg. In some embodiments, the compound of formula 1, or a pharmaceutically-acceptable salt thereof, is administered once a day. In some embodiments, the compound of formula 1, or a pharmaceutically-acceptable salt thereof, is administered twice a day. In some embodiments, the compound of formula 1, or a pharmaceutically-acceptable salt thereof, is administered at twice the dose on the first day.

In some embodiments, the administration of the compound of formula 1, or a pharmaceutically-acceptable salt thereof, results in a plasma AUC₀₋₂₄ under 500 ng*hr/mL, or under 250 ng*hr/mL, or under 100 ng*hr/mL, or under 50 ng*hr/mL.

In some embodiments, the administration of the compound of formula 1, or a pharmaceutically-acceptable salt thereof, results in a Tₘₐₓ between 0.5 hr and 4 hr, or between 0.5 and 2 hr, or a Tₘₐₓ of about 1 hr.

In some embodiments, the patient is symptomatic. In some embodiments, the patient is hospitalized. In some embodiments, the patient requires supplemental oxygen. In some embodiments, the patient requires supplemental oxygen, invasive mechanical ventilation, or extracorporeal membrane oxygenation (ECMO). In some embodiments, the patient has acute lung injury associated with COVID-19.

In some embodiments, the patient is 12 years old or older. In some embodiments, the patient is under 12 years old. In some embodiments, the patient is a pediatric patient two years of age or older.

In some embodiments, the patient has mild to moderate COVID-19. In some embodiments, the patient has severe COVID-19. In some embodiments, the patient is at high risk for progressing to severe COVID-19 and/or hospitalization.

In some embodiments, the method results in an improvement in the levels of Receptor for Advanced Glycation End-products (RAGE) in the patient. In some embodiments, the method results in a decrease in the levels of Receptor for Advanced Glycation End-products (RAGE) in the patient. In some embodiments, the method results in a decrease in lung injury to the patient.

In some embodiments, the method results in a decreased time to hospital discharge for the patient.

In some embodiments, the method results in an improvement in the levels of C-reactive protein (CRP) in the patient. In some embodiments, the method results in a decrease in the levels of C-reactive protein (CRP) in the patient. In some embodiments, the method results in an improvement in the levels of IL-6 in the patient. In some embodiments, the method results in a decrease in the levels of IL-6 in the patient. In some embodiments, the method results in an improvement in the levels of IFNγ in the patient. In some embodiments, the method results in a decrease in the levels of IFNγ in the patient. In some embodiments, the method results in an improvement in the levels of IP-10 in the patient. In some embodiments, the method results in a decrease in the levels of IP-10 in the patient. In some embodiments, the method results in a decrease in the levels of IL-10 in the patient. In some embodiments, the method results in a decrease in the levels of MCP-1 in the patient. In some embodiments, the method results in an improvement in the modified Borg Dyspnea Score for the patient.

In some embodiments, the method results in an increase in oxygen levels in the blood of the patient. In some embodiments, the method results in a decrease in the need for supplemental oxygen for the patient.

In some embodiments, the method results in a decrease in the mortality risk for the patient.

In some embodiments, the method results in a decrease in hospitalization time for the patient. In some embodiments, the method results in a decrease in time in the ICU for a patient.

In some embodiments, the method results in an increase in the number of RFDs (Respiratory failure-free days) for the patient. In some embodiments, the method results in an increase in the number of days without supplemental oxygen for the patient.

In some embodiments, the method results in a decreased time to recovery.

In some embodiments, the method comprises administering one or more additional therapeutic agents or treatments to the patient. In some embodiments, the patient receives standard of care co-treatment. In some embodiments, the patient is also treated with corticosteroids. In some embodiments, the patient is also treated with dexamethasone. In some embodiments, the patient is also treated with remdesivir.

In some embodiments, the patient suffers from hypertension and/or diabetes.

In some embodiments, the patient suffers from moderate COVID-19 when treatment with compound 1, or a pharmaceutically acceptable salt thereof, is initiated. In some embodiments, the patient suffers from severe COVID-19 when treatment with compound 1, or a pharmaceutically acceptable salt thereof, is initiated.

In some embodiments, the method results in an increase in the number of ventilator-free days (VFDs).

In some embodiments, the patient has COVID-19 associated acute lung injury. In some embodiments, the patient requires supplemental oxygen when admitted (e.g. to a hospital). In some embodiments, the patient requires supplemental oxygen but is not on ventilation or high-flow oxygen when admitted. In some embodiments, the patient requires invasive mechanical ventilation or extracorporeal membrane oxygenation when admitted. In some embodiments, the patient is on non-invasive ventilation or high-flow oxygen devices when admitted.

In some embodiments, the method prevents, reduces, or resolves acute lung injury and/or acute respiratory distress syndrome caused by COVID-19. In some embodiments, the method prevents, reduces, or stops a cytokine storm caused by COVID-19. In some embodiments, the method results in removal of the patient from ventilation or oxygen supplementation.

Also provided herein are uses of compound 1, or a pharmaceutically-acceptable salt thereof, for treating a patient infected with a coronavirus and having a baseline level of CRP below 150 mg/L and uses of compound 1, or a pharmaceutically-acceptable salt thereof, for the manufacture of a medicament useful to treat a patient infected with a coronavirus and having a baseline level of CRP below 150 mg/L.

Also provided herein is a method of treating a patient suffering from an inflammatory respiratory disease associated with an elevation in CRP levels, wherein the patient has a baseline level of CRP below 150 mg/L comprising administering to the patient a compound of formula 1:

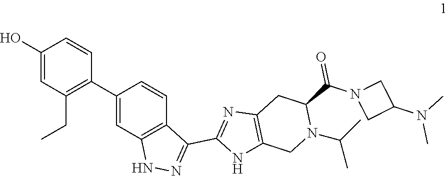 [SMILES: CCc1cc(O)ccc1-c1ccc2c(-c3nc4c([nH]3)CN(C(C)C)[C@H](C(=O)N3CC(N(C)C)C3)C4)n[nH]c2c1]

or a pharmaceutically-acceptable salt thereof.

Also provided herein is a method of treating a patient infected with influenza and having a baseline level of CRP below 150 mg/L comprising administering to the patient a compound of formula 1:

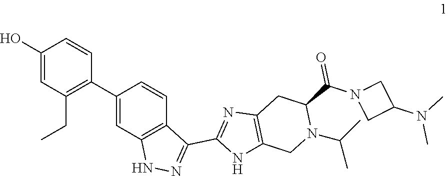 [SMILES: CCc1cc(O)ccc1-c1ccc2c(-c3nc4c([nH]3)CN(C(C)C)[C@H](C(=O)N3CC(N(C)C)C3)C4)n[nH]c2c1]

or a pharmaceutically-acceptable salt thereof. In some embodiments, the patient has influenza A. In some embodiments, the patient has influenza B. In some embodiments, the patient has influenza C. In some embodiments, the patient has influenza D.

Also provided herein is a method of treating a patient infected with a coronavirus and having a baseline level of CRP below 150 mg/L comprising administering to the patient a JAK inhibitor, or a pharmaceutically-acceptable salt thereof. In some embodiments, the JAK inhibitor inhibits JAK1, JAK2, JAK3, TYK2, and combinations thereof. In some embodiments, the JAK inhibitor inhibits JAK1, JAK2, JAK3, and TYK2. In some embodiments, the JAK inhibitor is administered by inhalation. In some embodiments, the JAK inhibitor is administered by nebulized inhalation. In some embodiments, the JAK inhibitor is tofacitinib, ruxolitinib, or baricitinib.

Chemical Structures

Chemical structures are named herein according to IUPAC conventions as implemented in ChemDraw software (PerkinElmer, Inc., Cambridge, Mass.). Compound 1 is designated as (S)-(3-(dimethylamino)azetidin-1-yl)(2-(6-(2-ethyl-4-hydroxyphenyl)-1H-indazol-3-yl)-5-isopropyl-4,5,6,7-tetrahydro-3H-imidazo[4,5-c]pyridin-6-yl)methanone.

Furthermore, the imidazo portion of the tetrahydroimidazopyridine moiety exists in tautomeric forms, illustrated below for a fragment of compound 1

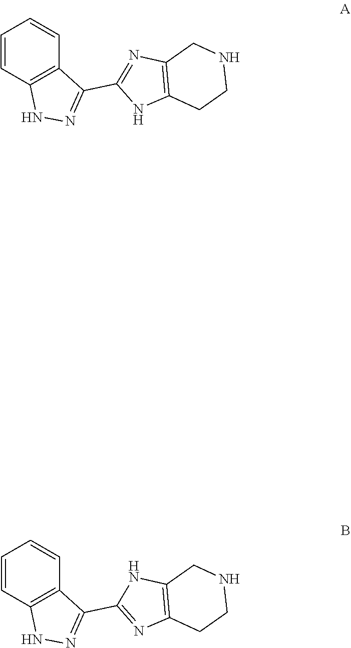 [SMILES: c1ccc2c(-c3nc4c([nH]3)CCNC4)n[nH]c2c1 ; c1ccc2c(-c3nc4c([nH]3)CNCC4)n[nH]c2c1]

According to the IUPAC convention, these representations give rise to different numbering of the atoms of the imidazole portion: (1H-indazol-3-yl)-4,5,6,7-tetrahydro-1H-imidazo[4,5-c]pyridine (structure A) vs. (1H-indazol-3-yl)-4,5,6,7-tetrahydro-3H-imidazo[4,5-c]pyridine (structure B). It will be understood that although structures are shown, or named, in a particular form, the disclosure also includes the tautomer thereof.

Compound 1 may exist as a pure enantiomer or as an enriched mixture. The depiction or naming of a particular stereoisomer means the indicated stereocenter has the designated stereochemistry with the understanding that minor amounts of other stereoisomers may also be present unless otherwise indicated, provided that the utility of the depicted or named compound is not eliminated by the presence of another stereoisomer.

Compound 1 also contains several basic groups (e.g., amino groups) and therefore, such compound can exist as the free base or in various salt forms, such a mono-protonated salt form, a di-protonated salt form, a tri-protonated salt form, or mixtures thereof. All such forms are included within the scope of this disclosure, unless otherwise indicated.

This disclosure also includes isotopically-labeled compounds of formula 1, i.e., compounds of formula 1 where one or more atom has been replaced or enriched with an atom having the same atomic number but an atomic mass different from the atomic mass that predominates in nature. Examples of isotopes that may be incorporated into a compound of formula 1 include, but are not limited to, ²H, ³H, ¹¹C, ¹³C, ¹⁴C, ¹³N, ¹⁵N, ¹⁵O, ¹⁷O, and ¹⁸O.

### Definitions

When describing this disclosure including its various aspects and embodiments, the following terms have the following meanings, unless otherwise indicated.

The term “about” means ±5 percent of the specified value.

The term “AUC₀₋₂₄” means the Area under the plasma concentration versus time curve, from time zero to 24 hours postdose.

The term “AUC_{0-∞}” means Area under the plasma concentration-time curve from time 0 extrapolated to infinity.

The term “Cₘₐₓ” means maximum observed plasma concentration.

The term “PK” means pharmacokinetic.

The term “Tₘₐₓ” means time to maximum plasma concentration.

The term “t_{1/2}” means apparent terminal elimination half-life.

The term “therapeutically effective amount” means an amount sufficient to effect treatment when administered to a patient in need of treatment.

The term “treating” or “treatment” means ameliorating or suppressing the medical condition, disease, or disorder being treated in a patient (particularly a human); or alleviating the symptoms of the medical condition, disease, or disorder.

The term “preventing” or “prevention” means treatment of a condition, disease, or condition that causes the clinical symptoms of the condition, disease, or condition not to develop.

The term “pharmaceutically acceptable salt” or term “pharmaceutically-acceptable salt” means a salt that is acceptable for administration to a patient or a mammal, such as a human (e.g., salts having acceptable mammalian safety for a given dosage regime). Representative pharmaceutically acceptable salts include salts of acetic, ascorbic, benzenesulfonic, benzoic, camphorsulfonic, citric, ethanesulfonic, edisylic, fumaric, gentisic, gluconic, glucoronic, glutamic, hippuric, hydrobromic, hydrochloric, isethionic, lactic, lactobionic, maleic, malic, mandelic, methanesulfonic, mucic, naphthalenesulfonic, naphthalene-1,5-disulfonic, naphthalene-2,6-disulfonic, nicotinic, nitric, orotic, pamoic, pantothenic, phosphoric, succinic, sulfuric, tartaric, p-toluenesulfonic and xinafoic acid, and the like.

The term “salt thereof” means a compound formed when the hydrogen of an acid is replaced by a cation, such as a metal cation or an organic cation and the like. For example, the cation can be a protonated form of a compound of formula 1, i.e. a form where one or more amino groups have been protonated by an acid. Typically, the salt is a pharmaceutically acceptable salt, although this is not required for salts of intermediate compounds that are not intended for administration to a patient.

### Pharmaceutical Compositions

Compound 1 and pharmaceutically-acceptable salts thereof are typically used in the form of a pharmaceutical composition or formulation. Such pharmaceutical compositions may advantageously be administered to a patient by inhalation. In addition, pharmaceutical compositions may be administered by any acceptable route of administration including, but not limited to, oral, rectal, nasal, topical (including transdermal) and parenteral modes of administration.

Provided herein are pharmaceutical compositions comprising a pharmaceutically-acceptable carrier or excipient and compound 1, where, as defined above, “compound 1” means compound 1, or a pharmaceutically-acceptable salt thereof. Optionally, such pharmaceutical compositions may contain other therapeutic and/or formulating agents if desired. When discussing compositions and uses thereof, compound 1 may also be referred to herein as the “active agent.”

The pharmaceutical compositions of the disclosure typically contain a therapeutically effective amount of compound 1. Those skilled in the art will recognize, however, that a pharmaceutical composition may contain more than a therapeutically effective amount, i.e., bulk compositions, or less than a therapeutically effective amount, i.e., individual unit doses designed for multiple administration to achieve a therapeutically effective amount, or an amount sufficient to effect a desired biological effect such as decreasing the viral load of a coronavirus.

Typically, such pharmaceutical compositions will contain from about 0.01 to about 95% by weight of the active agent; including, for example, from about 0.05 to about 30% by weight; and from about 0.1% to about 10% by weight of the active agent.

Any conventional carrier or excipient may be used in the pharmaceutical compositions comprising compound 1. The choice of a particular carrier or excipient, or combinations of carriers or excipients, will depend on the mode of administration being used to treat a particular patient or type of medical condition or disease state. In this regard, the preparation of a suitable pharmaceutical composition for a particular mode of administration is well within the scope of those skilled in the pharmaceutical arts. Additionally, the carriers or excipients used in the pharmaceutical compositions of this disclosure are commercially-available. By way of further illustration, conventional formulation techniques are described in Remington: The Science and Practice of Pharmacy, 20th Edition, Lippincott Williams & White, Baltimore, Md. (2000); and H. C. Ansel et al., Pharmaceutical Dosage Forms and Drug Delivery Systems, 7th Edition, Lippincott Williams & White, Baltimore, Md. (1999).

Representative examples of materials which can serve as pharmaceutically acceptable carriers include, but are not limited to, the following: sugars, such as lactose, glucose and sucrose; starches, such as corn starch and potato starch; cellulose, such as microcrystalline cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical compositions.

Pharmaceutical compositions are typically prepared by thoroughly and intimately mixing or blending the active agent with a pharmaceutically-acceptable carrier and one or more optional ingredients. The resulting uniformly blended mixture can then be shaped or loaded into tablets, capsules, pills and the like using conventional procedures and equipment.

In one aspect, the pharmaceutical composition is suitable for inhaled administration. Pharmaceutical compositions for inhaled administration are typically in the form of an aerosol or a powder. Such compositions are generally administered using inhaler delivery devices, such as a dry powder inhaler (DPI), a metered-dose inhaler (MDI), a nebulizer inhaler, or a similar delivery device.

In a particular embodiment, the pharmaceutical composition is administered by inhalation using a dry powder inhaler Such dry powder inhalers typically administer the pharmaceutical composition as a free-flowing powder that is dispersed in a patient's air-stream during inspiration. In order to achieve a free-flowing powder composition, the therapeutic agent is typically formulated with a suitable excipient such as lactose, starch, mannitol, dextrose, polylactic acid (PLA), polylactide-co-glycolide (PLGA) or combinations thereof. Typically, the therapeutic agent is micronized and combined with a suitable carrier to form a composition suitable for inhalation.

A representative pharmaceutical composition for use in a dry powder inhaler comprises lactose and compound 1 in micronized form. Such a dry powder composition can be made, for example, by combining dry milled lactose with the therapeutic agent and then dry blending the components. The composition is then typically loaded into a dry powder dispenser, or into inhalation cartridges or capsules for use with a dry powder delivery device.

Dry powder inhaler delivery devices suitable for administering therapeutic agents by inhalation are described in the art and examples of such devices are commercially available. For example, representative dry powder inhaler delivery devices or products include Aeolizer (Novartis); Airmax (IVAX); ClickHaler (Innovata Biomed); Diskhaler (GlaxoSmithKline); Diskus/Accuhaler (Glaxo SmithKline); Ellipta (Glaxo SmithKline); Easyhaler (Orion Pharma); Eclipse (Aventis); FlowCaps (Hovione); Handihaler (Boehringer Ingelheim); Pulvinal (Chiesi); Rotahaler (GlaxoSmithKline); SkyeHaler/Certihaler (SkyePharma); Twisthaler (Schering-Plough); Turbuhaler (AstraZeneca); Ultrahaler (Aventis); and the like.

In another particular embodiment, the pharmaceutical composition is administered by inhalation using a metered-dose inhaler Such metered-dose inhalers typically discharge a measured amount of a therapeutic agent using a compressed propellant gas. Accordingly, pharmaceutical compositions administered using a metered-dose inhaler typically comprise a solution or suspension of the therapeutic agent in a liquefied propellant. Any suitable liquefied propellant may be employed including hydrofluoroalkanes (HFAs), such as 1,1,1,2-tetrafluoroethane (HFA 134a) and 1,1,1,2,3,3,3-heptafluoro-n-propane, (HFA 227); and chlorofluorocarbons, such as CC13F. In a particular embodiment, the propellant is hydrofluoroalkanes. In some embodiments, the hydrofluoroalkane formulation contains a co-solvent, such as ethanol or pentane, and/or a surfactant, such as sorbitan trioleate, oleic acid, lecithin, and glycerin.

A representative pharmaceutical composition for use in a metered-dose inhaler comprises from about 0.01% to about 5% by weight of compound 1; from about 0% to about 20% by weight ethanol; and from about 0% to about 5% by weight surfactant; with the remainder being an HFA propellant. Such compositions are typically prepared by adding chilled or pressurized hydrofluoroalkane to a suitable container containing the therapeutic agent, ethanol (if present) and the surfactant (if present). To prepare a suspension, the therapeutic agent is micronized and then combined with the propellant. The composition is then loaded into an aerosol canister, which typically forms a portion of a metered-dose inhaler device.

Metered-dose inhaler devices suitable for administering therapeutic agents by inhalation are described in the art and examples of such devices are commercially available. For example, representative metered-dose inhaler devices or products include AeroBid Inhaler System (Forest Pharmaceuticals); Atrovent Inhalation Aerosol (Boehringer Ingelheim); Flovent (GlaxoSmithKline); Maxair Inhaler (3M); Proventil Inhaler (Schering); Serevent Inhalation Aerosol (GlaxoSmithKline); and the like.

In another particular aspect, the pharmaceutical composition is administered by inhalation using a nebulizer inhaler. Such nebulizer devices typically produce a stream of high velocity air that causes the pharmaceutical composition to spray as a mist that is carried into the patient's respiratory tract. Accordingly, when formulated for use in a nebulizer inhaler, the therapeutic agent can be dissolved in a suitable carrier to form a solution. Alternatively, the therapeutic agent can be micronized or nanomilled and combined with a suitable carrier to form a suspension.

A representative pharmaceutical composition for use in a nebulizer inhaler comprises a solution or suspension comprising from about 0.05 μg/mL to about 20 mg/mL of compound 1 and excipients compatible with nebulized formulations. In one embodiment, the solution has a pH of about 3 to about 8.

Nebulizer devices suitable for administering therapeutic agents by inhalation are described in the art and examples of such devices are commercially available. For example, representative nebulizer devices or products include the Respimat Softmist Inhalaler (Boehringer Ingelheim); the AERx Pulmonary Delivery System (Aradigm Corp.); the PARI LC Plus Reusable Nebulizer (Pari GmbH); and the like.

In yet another aspect, the pharmaceutical compositions of the disclosure may alternatively be prepared in a dosage form intended for oral administration. Suitable pharmaceutical compositions for oral administration may be in the form of capsules, tablets, pills, lozenges, cachets, dragees, powders, granules; or as a solution or a suspension in an aqueous or non-aqueous liquid; or as an oil-in-water or water-in-oil liquid emulsion; or as an elixir or syrup; and the like; each containing a predetermined amount of compound 1 as an active ingredient.

When intended for oral administration in a solid dosage form, the pharmaceutical compositions comprising compound 1 will typically comprise the active agent and one or more pharmaceutically-acceptable carriers, such as sodium citrate or dicalcium phosphate. Optionally or alternatively, such solid dosage forms may also comprise: fillers or extenders, binders, humectants, solution retarding agents, absorption accelerators, wetting agents, absorbents, lubricants, coloring agents, and buffering agents. Release agents, wetting agents, coating agents, sweetening, flavoring and perfuming agents, preservatives and antioxidants can also be present in the pharmaceutical compositions of the disclosure.

Alternative formulations may also include controlled release formulations, liquid dosage forms for oral administration, transdermal patches, and parenteral formulations. Conventional excipients and methods of preparation of such alternative formulations are described, for example, in the reference by Remington, supra.

The following non-limiting examples illustrate representative pharmaceutical compositions of the present disclosure.

Dry Powder Composition

Micronized compound 1 (1 g) is blended with milled lactose (25 g). This blended mixture is then loaded into individual blisters of a peelable blister pack in an amount sufficient to provide between about 0.1 mg to about 4 mg of compound 1 per dose. The contents of the blisters are administered using a dry powder inhaler.

Dry Powder Composition

Micronized compound 1 (1 g) is blended with milled lactose (20 g) to form a bulk composition having a weight ratio of compound to milled lactose of 1:20. The blended composition is packed into a dry powder inhalation device capable of delivering between about 0.1 mg to about 4 mg of compound 1 per dose.

Metered-Dose Inhaler Composition

Micronized compound 1 (10 g) is dispersed in a solution prepared by dissolving lecithin (0.2 g) in demineralized water (200 mL). The resulting suspension is spray dried and then micronized to form a micronized composition comprising particles having a mean diameter less than about 1.5 μm. The micronized composition is then loaded into metered-dose inhaler cartridges containing pressurized 1,1,1,2-tetrafluoroethane in an amount sufficient to provide about 0.1 mg to about 4 mg of compound 1 per dose when administered by the metered dose inhaler.

Nebulizer Composition

Compound 1 (25 mg) is dissolved in a solution containing 1.5-2.5 equivalents of hydrochloric acid, followed by addition of sodium hydroxide to adjust the pH to 3.5 to 5.5 and 3% by weight of glycerol. The solution is stirred well until all the components are dissolved. The solution is administered using a nebulizer device that provides about 0.1 mg to about 4 mg of compound 1 per dose.

Compound 1, or a pharmaceutically acceptable salt thereof, will typically be administered in a single daily dose or in multiple doses per day, although other forms of administration may be used. The amount of active agent administered per dose or the total amount administered per day will typically be determined by a physician, in the light of the relevant circumstances, including the condition to be treated, the chosen route of administration, the actual compound administered and its relative activity, the age, weight, and response of the individual patient, the severity of the patient's symptoms, and the like.

### Utility

Compound 1 is a potent inhibitor of the JAK family of enzymes: JAK1, JAK2, JAK3, and TYK2, and a potent inhibitor of pro-inflammatory and pro-fibrotic cytokines. It has been recognized that the broad anti-inflammatory effect of systemically available JAK inhibitors could suppress normal immune cell function, potentially leading to an increased risk of infections. By contrast, compound 1 enables the delivery of a potent anti-cytokine agent directly to the site of action of the respiratory disease, in the lung, while limiting systemic exposure.

Compound 1 was evaluated in a phase 1 clinical trial and dosed in humans through nebulized inhalation at 1 mg, 3 mg, and 10 mg for up to 7 days. The plasma Cₘₐₓ (maximum plasma concentration) values of the compound of formula 1 were found to be well under the binding-corrected JAK IC₅₀, i.e. the plasma concentration necessary to inhibit Janus kinases by 50%. The pharmacokinetics of inhaled compound 1 are consistent with low plasma exposures after inhaled administration. Maximal plasma exposures of compound 1 were about 20-fold and about 7-fold lower than the protein-adjusted JAK IC₅₀ at dose levels of 3 and 10 mg, respectively. Additionally, absolute NK cell counts were evaluated after multiple-dosing to assess the potential for systemic pharmacologic effects associated with JAK inhibition by compound 1. No reductions in NK cells were observed relative to baseline in participants receiving placebo or compound 1 at any dose level (1, 3, or 10 mg) explored in the study. The lack of reduction in NK cell counts is also consistent with the lack of systemic JAK inhibition. In contrast, marked reductions in NK cell counts have been observed with systemic JAK inhibitors such as tofacitinib (Weinhold, K. J., et al., Reversibility of peripheral blood leukocyte phenotypic and functional changes after exposure to and withdrawal from tofacitinib, a Janus kinase inhibitor, in healthy volunteers. Clin Immunol. 191, 10-20, 2018). Other systemically mediated hematological changes associated with JAK inhibition, including neutrophil and hemoglobin reductions as well as lipid changes, were not observed with inhaled administration of compound 1. These results support a favorable safety and tolerability profile and PK below levels anticipated to exert systemic effects.

Human coronavirus is a common respiratory pathogen and typically induces mild upper respiratory disease. The two highly pathogenic viruses, Severe Acute Respiratory Syndrome associated-Coronavirus (SARS-CoV-1) and Middle East Respiratory Syndrome-associated Coronavirus (MERS-CoV), caused severe respiratory syndromes resulting in more than 10% and 35% mortality, respectively (Assiri et al., N Engl J Med., 2013, 369, 407-1). The recent emergence of Coronavirus Disease 2019 (COVID-19) and the associated pandemic has created a global health care emergency. Similar to SARS-CoV-1 and MERS-CoV, a subset of patients (about 16%) can develop a severe respiratory illness manifested by acute lung injury (ALI) leading to ICU admission (about 5%), respiratory failure (about 6.1%) and death (Wang et al., JAMA, 2020, 323, 11, 1061-1069; Guan et al., N Engl J Med., 2020, 382, 1708-1720; Huang et al., The Lancet, 2020. 395 (10223), 497-506; Chen et al., The Lancet, 2020, 395(10223), 507-13). A subgroup of patients with COVID-19 appears to have a hyperinflammatory “cytokine storm” resulting in acute lung injury and acute respiratory distress syndrome (ARDS). This cytokine storm may also spill over into the systemic circulation and produce sepsis and ultimately, multi-organ dysfunction syndrome. The dysregulated cytokine signaling that appears in COVID-19 is characterized by increased expression of interferons (IFNs), interleukins (ILs), and chemokines, resulting in ALI and associated mortality.

Infection with mouse adapted strains of the 2003 SARS-CoV-1 and 2012 MERS-CoV, as well as a transgenic mouse expressing the human SARS-CoV-1 receptor hACE2 infected with human SARS-CoV-1, demonstrate elevations of JAK-dependent cytokines, such as IFNγ, IL-6, and IL-12, and downstream chemokines, such as chemokine (C-C motif) ligand 10 (CCL10), CCL2, and CCL7 (McCray et al., J Virol., 2007, 81(2), 813-21; Gretebeck et al., Curr Opin Virol. 2015, 13, 123-9.; Day et al., Virology. 2009, 395(2), 210-22. It was recently shown that similar to SARS-CoV-1 and MERS-CoV, patients with severe COVID-19 have elevated Th17, which can be driven by IL-6 and IL-23 via signal transducer and activator of transcription 3, STAT3 (Huang et al., Lancet 2020, 395, 497-506). Mouse Th17 cells produce large amounts of IL-17 in response to IL-23 which can be blocked with a JAK inhibitor (Wu et al., J Microbiol Immunol Infect., 2020, S1684118220300657). Though IFN responses can be protective in virus infection, there is evidence that a delayed response in humans contributes to virus-induced acute respiratory distress syndrome (Chen et al., Annu Rev Immunol., 2007, 25(1), 443-72) Similarly, mice deficient in the IFNα/β receptor IFNR1 are protected from lethal SARS-CoV-1 infection (Channappanavar R, Fehr A R, Vijay R, Mack M, Zhao J, Meyerholz D K, et al. Dysregulated Type I Interferon).

Concerns have also been raised about the potential increased risk for thromboembolism with systemic JAK inhibitors which is particularly concerning given observations of severe hypercoagulability in patients with COVID-19.

Compound 1, a lung-selective, inhaled pan-JAK inhibitor, addresses the shortcomings of oral JAK inhibitors by avoiding systemic immunosuppression, thromboembolisms, and additional infections that lead to worsened mortality.

Further, compound 1 has been used and can be used alone or in combination with standard of care including remdesivir and corticosteroids such as dexamethasone.

Compound 1 acts through a mechanism of action that can dampen the cytokine storm associated with a coronavirus infection.

As further detailed in the experimental section, compound 1 has been studied in a phase 2 clinical study in COVID patients. Although there was no statistically significant difference in RFDs from randomization through Day 28 between compound 1 and placebo in ITT (median: 21 vs. 21 days; p=0.61) and no difference in change from baseline at Day 7 in SaO₂/FiO₂ ratio, in a proportion of patients in each category of the 8-point Clinical Status scale, and proportion of patients alive and respiratory failure-free at Day 28, compound 1 demonstrated a favorable trend in improvement when compared to placebo for 28-day all-cause mortality (total number of deaths: 6 vs. 13, HR: 0.42, p=0.08) and time to recovery (median: 10 vs. 11 days, HR: 1.27, p=0.12).

Importantly, in a post-hoc analysis of patients with baseline CRP (n=201), in patients with CRP <150 mg/L (n=171), there was an improvement in those treated with compound 1 when compared to placebo in:
28-day all-cause mortality (total number of deaths: 1 vs 9, HR: 0.097, p=0.009).
time to recovery (median: 10 vs. 11 days, HR: 1.48, p=0.02).

In patients with CRP >150 mg/L (n=30), there was no difference in time to recovery or 28-day all-cause mortality between those treated with compound 1 or placebo.

Therefore, compound 1 is particularly adapted for the treatment of the subpopulation of patients having a CRP (C-reactive protein) baseline level below 150 mg/L.

C-reactive protein (CRP) is a protein found in blood plasma, whose circulating concentrations rise in response to inflammation. It is an acute-phase protein of hepatic origin that increases following interleukin-6 secretion by macrophages and T cells. In healthy adults, the normal concentrations of CRP varies between 0.8 mg/L and 3.0 mg/L. However, some healthy adults show elevated CRP at 10 mg/L.

Compound 1 was well-tolerated. Adverse events and serious adverse events occurred in 34.0% and 9.7% of patients treated with compound 1, and 41.2% and 15.7% of patients treated with placebo, respectively. Adverse events of liver abnormalities or disease occurred in 9.7% and 7.8% of patients treated with compound 1 and placebo, respectively. Serious infections and venous thromboembolism occurred in 1.0% and none of the patients treated with compound 1, and 2.0% and 4.9% in patients treated with placebo, respectively.

Plasma exposure of compound 1 was low and consistent with expectations for a lung-selective medicine.

Additionally, coronaviruses gain entry into host cells by fusing with cellular membranes, a step that is required for virus replication. Abelson kinase inhibitors have been reported to be potent inhibitors of SARS-CoV-1 and MERS-CoV fusion (Coleman et al., Journal of Virology, 2016, 90, 19, 8924-8933; Sisk et al., Journal of General Virology, 2018, 99, 619-630), supporting the fact that an Abelson kinase inhibitor could be useful for treating patients infected with a coronavirus by decreasing the viral load of the patient. Compound 1 has been shown to potently inhibit Abl2 in Assay 6.

Therefore, without being limited by this theory, compound 1 may be uniquely suited for the treatment of coronaviruses, as a compound which can be delivered selectively to the lungs, possesses pan-JAK inhibitory activity that can dampen the cytokine storm associated with COVID-19, and possesses Abelson kinase inhibitory activity that could decrease the viral load of the coronavirus in the patient.

Abl kinases have also been reported to have a positive role in regulating endothelial barrier function and vascular leak during acute lung injury. Therefore, compound 1, or a pharmaceutically acceptable salt thereof, may also work by strengthening endothelial cell-to-cell contacts and promoting endothelial cell adhesion to the extracellular matrix.

Under another theory, the potential ability of compound 1 to directly affect the coronavirus may counter-balance or mitigate a possible increase in local viral replication caused by the use of a compound causing immune suppression.

It has also been reported that the ability of neutrophils to form neutrophil extracellular traps (NETs) may contribute to organ damage and mortality in COVID-19 patients (Barnes et al., J. Exp. Med., 2020, 217, 6, e20200652, 1-7). Aberrant NET formation has been linked to pulmonary diseases, thrombosis, mucous secretions in the airways, and cytokine production. Therefore, compound 1, or a pharmaceutically-acceptable salt thereof, may be useful to (a) block or inhibit neutrophilia and/or the formation of neutrophil extracellular traps (NETs) in a patient infected with a coronavirus, (b) decrease the risk of thrombosis in a patient infected with a coronavirus, and/or (c) decrease the incidence of thrombosis in a patient population infected with a coronavirus.

Multisystem inflammatory syndrome in children (MIS-C) is a condition where different body parts can become inflamed, including the heart, lungs, kidneys, brain, skin, eyes, or gastrointestinal organs. MIS-C has been associated with exposure to COVID-19 and appears to be a rare but serious complication associated with COVID-19. MIS-C is associated with inflammation of the lungs. Therefore, compound 1 is expected to be useful in preventing or treating MIS-C.

Further, respiratory epithelial cell death by influenza virus infection is responsible for the induction of inflammatory responses. It has been shown that Influenza A virus infection triggers pyroptosis and apoptosis of respiratory epithelial cells through the Type I Interferon signaling pathway (Lee et al., Journal of Virology, 2018, 92, 14, e00396-18). The type I interferon (IFN)-mediated JAK-STAT signaling pathway promotes the switch from apoptosis to pyroptosis by inhibiting apoptosis possibly through the induced expression of the Bcl-xL anti-apoptotic gene. Further, the inhibition of JAK-STAT signaling repressed pyroptosis but enhanced apoptosis in infected PL16T cells. This suggests that the type I IFN signaling pathway plays an important role to induce pyroptosis but represses apoptosis in the respiratory epithelial cells to initiate proinflammatory responses against influenza virus infection. Accordingly, the compound of formula 1, or a pharmaceutically-acceptable salt thereof, is expected to be useful to treat influenza patients. Based on its mechanism of action, the compound of formula 1, or a pharmaceutically-acceptable salt thereof, is expected to prevent or treat inflammation in the lungs and/or ALI and/or ARDS in influenza patients.

Combination Therapy

Compound 1, or a pharmaceutically acceptable salt thereof, may be used in combination with one or more additional therapeutic agents or treatments which act by the same mechanism or by different mechanisms to treat a disease. The different therapeutic agents or treatments may be administered sequentially or simultaneously, in separate compositions or in the same composition. Useful classes of therapeutical agents for combination therapy include, but are not limited to, an IL-6 inhibitor, an IL-6 receptor antagonist, an IL-6 receptor agonist, an IL-2 inhibitor, an antiviral, an anti-inflammatory drug, a sodium-glucose cotransporter 2 inhibitor, a vaccine, an ACE2 inhibitor, an antibiotic, an antiparasitic, a sphingosine 1-phosphate receptor modulator, a TMPRSS2 inhibitor, a TNF alpha inhibitor, an anti-TNF, a membrane haemagglutinin fusion inhibitor, an inhibitor of the terminal glycosylation of ACE2, a CCR5 inhibitor, stem cells, allogeneic mesenchymal stem cells, CRISPR therapy, CAR-T therapy, TCR-T therapy, a virus-neutralizing monoclonal antibody, a protease inhibitor, a SARS-CoV-2 antibody, a siRNA, a plasma-derived immunoglobulin therapy, a S-protein modulator, a PLX stem cell therapy, chimeric humanized virus suppressing factor, multipotent adult progenitor cell therapy, an anti-viroporin, umbilical cord-derived mesenchymal stem cells, a polymerase inhibitor, autologous adipose-derived mesenchymal stem cells, an angiotensin converting enzyme 2 inhibitor, an immunoglobulin agonist, a nucleoside reverse transcriptase inhibitor, a cytotoxic T-lymphocyte protein-4 inhibitor, a lung surfactant associated protein D modulator, a protease inhibitor, a nuclear factor kappa B inhibitor, a xanthine oxidase inhibitor, an endoplasmin modulator, a CCL26 gene inhibitor, a TLR modulator, a TLR agonist, a TLR-2 agonist, a TLR-6 agonist, a TLR-9 agonist, a TLR-4 agonist, a TLR-7 agonist, a TLR-3 agonist, an opioid receptor antagonist, a moesin inhibitor, an angiotensin converting enzyme 2 modulator, a MEK protein kinase inhibitor, aCD40 ligand receptor agonist, a CD70 antigen modulator, an amyloid protein deposition inhibitor, an apolipoprotein gene stimulator, a bromodomain containing protein 2 inhibitor, a bromodomain containing protein 4 inhibitor, an IL-15 receptor agonist, an immunoglobulin gamma Fc receptor III agonist, a MEK-1 protein kinase inhibitor, a Ras gene inhibitor, an interferon beta ligand, a galectin-3 inhibitor, a heat shock protein inhibitor, an elongation factor 1 alpha 2 modulator, a VEGF-1 receptor modulator, an Angiotensin II AT-2 receptor agonist, a basigin inhibitor, a viral envelope glycoprotein inhibitor, a gelsolin stimulator, a trypsin inhibitor, a GM-CSF ligand inhibitor, a urokinase plasminogen activator inhibitor, a serine protease inhibitor, a PDE 3 inhibitor, a PDE 4 inhibitor, a C-reactive protein inhibitor, a chemokine CC22 ligand inhibitor, a GM-CSF receptor antagonist, an hemoglobin scavenger receptor antagonist, a metalloprotease-1 inhibitor, a metalloprotease-3 inhibitor, a metalloprotease inhibitor, a small inducible cytokine A17 ligand inhibitor, a VEGF gene inhibitor, a Coronavirus spike glycoprotein inhibitor, a nucleoprotein inhibitor, an ATP binding cassette transporter B5 modulator, a vimentin modulator, a stem cell antigen-1 inhibitor, a casein kinase II inhibitor, a complement C5a factor inhibitor, an aldose reductase inhibitor, a calpain-I inhibitor, a calpain-II inhibitor, a calpain-IX inhibitor, a proto-oncogene Mas agonist, a non-nucleoside reverse transcriptase inhibitor, an Interferon gamma ligand inhibitor, a CD4 modulator, a TGFB2 gene inhibitor, an Interleukin-1 beta ligand inhibitor, an inosine monophosphate dehydrogenase inhibitor, an angiotensin converting enzyme 2 stimulator, an adenosine A3 receptor agonist, a palmitoyl protein thioesterase 1 inhibitor, a Btk tyrosine kinase inhibitor, a NK1 receptor antagonist, an acetaldehyde dehydrogenase inhibitor, a CGRP receptor antagonist, a prostaglandin E synthase-1 inhibitor, a VIP receptor agonist, a nuclear factor kappa B gene modulator, a Grp78 calcium binding protein inhibitor, a Jun N terminal kinase inhibitor, a transferrin modulator, a p38 MAP kinase modulator, a CCR5 chemokine antagonist, a APOA1 gene stimulator, a bromodomain containing protein 2 inhibitor, a bromodomain containing protein 4 inhibitor, a BMP10 gene inhibitor, a BMP15 gene inhibitor, an adrenergic receptor antagonist, a human papillomavirus E6 protein modulator, a human papillomavirus E7 protein modulator, a Ca2+ release activated Ca2+ channel 1 inhibitor, an amyloid protein deposition inhibitor, a gamma-secretase inhibitor, a 2,5-Oligoadenylate synthetase stimulator, an Interferon type I receptor agonist, a ribonuclease stimulator, a S phase kinase associated protein 2 inhibitor, a dehydropeptidase-1 modulator, a calcium channel modulator, a signal transducer CD24 modulator, a cyclin E inhibitor, a cyclin-dependent kinase-2 inhibitor, a cyclin-dependent kinase-5 inhibitor, a cyclin-dependent kinase-9 inhibitor, a GM-CSF ligand inhibitor, an Interferon receptor modulator, an Interleukin-29 ligand, a cyclin-dependent kinase-7 inhibitor, a MCL1 gene inhibitor, a complement C5 factor inhibitor, an heparin agonist, an exo-alpha sialidase modulator, a muscarinic receptor antagonist, an IL-8 receptor antagonist, a vitamin D3 receptor agonist, a high mobility group protein B1 inhibitor, a CASP8-FADD-like regulator inhibitor, an ecto NOX disulfide thiol exchanger 2 inhibitor, a sphingosine kinase inhibitor, a sphingosine-1-phosphate receptor-1 antagonist, a stimulator of interferon genes protein stimulator, a topoisomerase inhibitor, an X-linked inhibitor of apoptosis protein inhibitor, an angiopoietin ligand-2 inhibitor, a neuropilin 2 inhibitor, a listeriolysin stimulator, an Interferon gamma receptor agonist, a MAPK gene modulator, a GM-CSF ligand inhibitor, an immunoglobulin G1 modulator, an immunoglobulin kappa modulator, a kallikrein modulator, a mannan-binding lectin serine protease inhibitor, an ubiquitin modulator, an IL12 gene stimulator, a xanthine oxidase inhibitor, a dihydroorotate dehydrogenase inhibitor, an IL-17 antagonist, a MAP kinase inhibitor, a PARP inhibitor, a poly ADP ribose polymerase 1 inhibitor, a poly ADP ribose polymerase 2 inhibitor, a dipeptidyl peptidase I inhibitor, a Btk tyrosine kinase inhibitor, a type I IL-1 receptor antagonist, an exportin 1 inhibitor, a hyaluronidase inhibitor, a sodium glucose transporter-2 inhibitor, a dihydroceramide delta 4 desaturase inhibitor, a sphingosine kinase 2 inhibitor, an Interferon beta ligand, an ICAM-1 stimulator, a TNF antagonist, a vascular cell adhesion protein 1 agonist, a COVID19 Spike glycoprotein modulator, a complement C1s subcomponent inhibitor, a NMDA receptor epsilon 2 subunit inhibitor, a tankyrase-1 inhibitor, a protein translation initiation inhibitor, a sigma receptor modulator, a sigmaR1 receptor modulator, a sigmaR2 receptor modulator, an antihistamine, an anti-05aR, a RNAi. a corticosteroid, a BCR-ABL a tyrosine kinase inhibitor, a colony stimulating factor, an inhibitor of tissue factor (TF), a recombinant granulocyte macrophage colony-stimulating factor (GM-CSF), a Gardos channel blocker, a heat-shock protein 90 (Hsp90) inhibitor, an alpha blocker, a cap binding complex modulator, a LSD1 inhibitor, a CRAC channel inhibitor, a RNA polymerase inhibitor, a CCR2 antagonist, a DHODH inhibitor, a blood thinner, an anti-coagulant, a factor Xa inhibitor, a SSRI, a SNRI, a sigma-1 receptor activator, a beta-blocker, a caspase inhibitor, a serine protease inhibitor, an IL-23A modulator, a NLRP3 inhibitor, an Angiopoietin-Tie2 signaling pathway modulator, a mannan-binding lectin-associated serine protease-2 modulator, a PDE4 inhibitor, a Vasoactive Intestinal Polypeptide, a microtubule depolymerization agent, a (PD)-1 checkpoint inhibitor, an Axl kinase inhibitor, a (PD)-1/PD-L1 checkpoint inhibitor, a PD-L1 checkpoint inhibitor, a T-cell CD61 receptor modulator, a Factor XIIa antagonist, an oral spleen tyrosine kinase (SYK) inhibitor, a CK2 inhibitor, a NMDA receptor antagonist, a SK2 inhibitor, an antiandrogen, and a tankyrase-2 inhibitor.

Specific therapeutical agents that may be used in combination with compound 1 include, but are not limited to cidofovir triphosphate, cidofovir, abacavir, ganciclovir, stavudine triphosphate, 2′-O-methylated UTP, desidustat, ampion, trans sodium crocetinate, CT-P59, Ab8, heparin, apixaban, GC373, GC376, Oleandrin, GS-441524, sertraline, Lanadelumab, zilucoplan, abatacept, CLBS119, Ranitidine, Risankizumab, AR-711, AR-701, MP0423, bempegaldesleukin, melatonin, carvedilol, mercaptopurine, paroxetine, casirivimab, imdevimab, ADG20, emricasan, dapansutrile, ceniciviroc infliximab, DWRX2003, AZD7442, MAN-19, LAU-7b, niclosamide, ANA001, fluvoxamine, narsoplimab, Sarconeos, GIGA-2050, VERU-111, REGN-COV2, icatibant, cenicriviroc, NTR-441, LAM-002A, oseltamivir, VHH72-Fc, MK-4482, EB05, OB-002, CM-4620-IE, IMU-838, SNG001, NT-17, BOLD-100, WP1122, itolizumab, PB1046, fostamatinib, colchicine, M5049, EDP1815, ABX464, CPI-006, azelastine, garadacimab, silmitasertib, lopinavir, ritonavir, remdesivir, cloroquine, hydrochloroquine, convalescent plasma transfusion, azithromycin, tocilizumab, famotidine, sarilumab, interferon beta, interferon beta-1a, interferon beta-1b, peginterferon lambda-1a, favipiravir, ASDC-09, dapagliflozin, CD24Fc, ribavirin, umifenovir, nitric oxide, APN01, teicoplanin, oritavancin, dalbavancin, monensin, ivermectin, darunavir, cobicistat, fingolimod, camostat, galidesicir, thalomide, leronlimab, remestemcel-L, canakinumab, TAK-888, azvudine, BPI-002, AT-100, T-89, Neumifil, GreMERSfi, liposomal curcumin, OYA-1, oxypurinol, mosedipimod, PUL-042, naltrexone, metenkefalin, COVID-EIG, TNX-1800, ATR-002, 177Lu-EC-Amifostine, 99mTc-EC-Amifostine, apabetalone, STI-6991, STI-4398, antroquinonol, ZIP-1642, DPX-COVID-19, belapectin, GX-19, AdCOVID, siltuximab, IBIO-200, plitidepsin, C-21, meplazumab, pathogen-specific aAPC, LV-SMENP-DC, ARMS-I, rhu-pGSN, PRTX-007, CK-0802, namilumab, upamostat, NI-007, COVID-HIG, CYNK-001, Nafamostat, brilacidin, mavrilimumab, IPT-001, PittCoVacc, allo-APZ2-Covid19, ENU-200, VIR-7832, VIR-7831, pritumumab, Ampion, TZLS-501, sodium pyruvate, silmitasertib, CoroFlu, BDB-1, AT-001, BLD-2660, 20-hydroxyecdysone, IFX-1, elsulfavirine, emapalumab, CEL-1000, trabedersen, VBI-2901, ASC-09, TJM-2, RPH-104, tranexamic acid, WP-1122, olokizumab, APN-01, danoprevir, piclidenoson, FW-1022, CORAVAX, Lamellasome COVID-19, COVID-19 XWG-03, EIDD-2801, AVM-0703, DC-661, acalabrutinib, bitespiramycin, Allocetra, tradipitant, bacTRL-Tri, Ad5-nCoV, EPV-CoV19, ADX-629, vazegepant, mercaptamine, sonlicromanol, aviptadil, fenretinide, IT-139, nitazoxanide, apabetalone, lucinactant, bacTRL-Spike, SAB-185, NVX-CoV2373, CM-4620, INO-4800, eicosapentaenoic acid, itanapraced, rintatolimod, XAV-19, niclosamide, ciclesonide, DAS181, ORBCEL-C, Metablok, dantrolene, CD24-IgFc, fadraciclib, gimsilumab, seliciclib, Cyto-MSC, ST-266, MRx-0004, ravulizumab, tafoxiparin, DAS-181, BMS-986253, cholecalciferol, nafamostat, ChAdOx1 nCoV-19, idronoxil, LY-3127804, ATYR-1923, VPM-1002, Mycobacterium w, lenzilumab, Polyoxidonium, conestat alfa, ubiquitin proteasome modulator, COVID-19 virus main protease Mpro inhibitor, mRNA-1273, clevudine, bucillamine, sodium meta-arsenite, vidofludimus, DARPin, COV-ENT-1, KTH-222, mefuparib, brensocatib, zanubrutinib, anakinra, selinexor, sarilumab, astodrimer, dapagliflozin propanediol, opaganib, BNT-162c2, BNT-162b2, BNT-162b1, BNT-162a1, ifenprodil, PIC1-01, 2X-121, zotatifin, aplidin, cloperastine, clemastine, dociparstat, avdoralimab, VIR-2703, ALN-COV, intravenous immunoglobulin (IVIg), apremilast, vicromax, baloxavir marboxil, emtricitabine, tenofovir, novaferon, secukinumab, valsartan, imatinib, omalizumab, leucine, sofosbuvir, alovudine, zidovudine, R-107, AB-201, sargramostim, LYT-100, senicapoc, fluvoxamine, aspirin, losartan, ADX-1612, ADX-629, sirikumab, otilimab, STI-1499, TR-C19, ABX-464, interferon alpha2b, arbidol, 5309, vafidemstat, AT-527, ibudilast, auxora, bemcentinib, eculizumab, JS016, FSD-201, LY-CoV555, avifavir, OP-101, RLF-100, DMX-200, 47D11, remsima, TYR1923, dexamethasone, EDP-1815, PTC29, rabeximod, foralumab, budesonide, molnupiravir, ensovibep, dalcetrapib, FSD201, pralatrexate, proxalutamide, clofazimine and merimepodib.

In some embodiments, compound 1, or a pharmaceutically acceptable salt thereof, is used in combination with an antiviral. In some embodiments, compound 1, or a pharmaceutically acceptable salt thereof, is used in combination with a corticosteroid. In some embodiments, compound 1, or a pharmaceutically acceptable salt thereof, is used in combination with an antiviral and a corticosteroid. In some embodiments, the antiviral is remdesivir. In some embodiments, the antiviral is favipiravir. In some embodiments, the corticosteroid is dexamethasone.

Also provided, herein, is a pharmaceutical composition comprising compound 1, or a pharmaceutically acceptable salt thereof, and one or more other therapeutic agents. The therapeutic agent may be selected from the class of agents specified above and from the list of specific agents described above. In some embodiments, the pharmaceutical composition is suitable for delivery to the lungs. In some embodiments, the pharmaceutical composition is suitable for inhaled or nebulized administration. In some embodiments, the pharmaceutical composition is a dry powder or a liquid composition.

Further, for all the methods disclosed herein, the methods comprise administering to the mammal, human or patient, compound 1, or a pharmaceutically acceptable salt thereof, and one or more other therapeutic agents.

When used in combination therapy, the agents may be formulated in a single pharmaceutical composition, or the agents may be provided in separate compositions that are administered simultaneously or at separate times, by the same or by different routes of administration. Such compositions can be packaged separately or may be packaged together as a kit. The two or more therapeutic agents in the kit may be administered by the same route of administration or by different routes of administration.

### Examples

Compound 1 was prepared as described in co-pending U.S. patent application Ser. No. 16/559,077 (US Pat. Pub. 2020/0071323), filed on Sep. 3, 2019 and in co-pending U.S. patent application Ser. No. 16/559,091 (US Pat. Pub. 2020/0071324), filed on Sep. 3, 2019.

### Biological Assays

Assay 1: Biochemical JAK Kinase Assays

A panel of four LanthaScreen JAK biochemical assays (JAK1, 2, 3 and Tyk2) were carried in a common kinase reaction buffer (50 mM HEPES, pH 7.5, 0.01% Brij-35, 10 mM MgCl₂, and 1 mM EGTA). Recombinant GST-tagged JAK enzymes and a GFP-tagged STAT1 peptide substrate were obtained from Life Technologies.

Serially diluted compounds were pre-incubated with each of the four JAK enzymes and the substrate in white 384-well microplates (Corning) at ambient temperature for 1 h. ATP was subsequently added to initiate the kinase reactions in 10 μL total volume, with 1% DMSO. The final enzyme concentrations for JAK1, 2, 3 and Tyk2 are 4.2 nM, 0.1 nM, 1 nM, and 0.25 nM respectively; the corresponding Km ATP concentrations used are 25 μM, 3 μM, 1.6 μM, and 10 μM; while the substrate concentration is 200 nM for all four assays. Kinase reactions were allowed to proceed for 1 hour at ambient temperature before a 10 μL preparation of EDTA (10 mM final concentration) and Tb-anti-pSTAT1 (pTyr701) antibody (Life Technologies, 2 nM final concentration) in TR-FRET dilution buffer (Life Technologies) was added. The plates were allowed to incubate at ambient temperature for 1 h before being read on the EnVision reader (Perkin Elmer). Emission ratio signals (520 nm/495 nm) were recorded and utilized to calculate the percent inhibition values based on DMSO and background controls.

For dose-response analysis, percent inhibition data were plotted vs. compound concentrations, and IC₅₀ values were determined from a 4-parameter robust fit model with the Prism software (GraphPad Software). Results were expressed as pIC₅₀ (negative logarithm of IC₅₀) and subsequently converted to pKᵢ (negative logarithm of dissociation constant, Ki) using the Cheng-Prusoff equation.

Test compounds having a lower Kᵢ value or higher pKᵢ value in the four JAK assays show greater inhibition of JAK activity.

Assay 2: Inhibition of IL-2 Stimulated pSTAT5 in Tall-1 T Cells

The potency of test compounds for inhibition of interleukin-2 (IL-2) stimulated STAT5 phosphorylation was measured in the Tall-1 human T cell line (DSMZ) using AlphaLisa. Because IL-2 signals through JAK1/3, this assay provides a measure of JAK1/3 cellular potency.

Phosphorylated STAT5 was measured via the AlphaLISA SureFire Ultra pSTAT5 (Tyr694/699) kit (PerkinElmer).

Human T cells from the Tall-1 cell line were cultured in a 37° C., 5% CO₂ humidified incubator in RPMI (Life Technologies) supplemented with 15% Heat Inactivated Fetal Bovine Serum (FBS, Life Technologies), 2 mM Glutamax (Life Technologies), 25 mM HEPES (Life Technologies) and 1×Pen/Strep (Life Technologies). Compounds were serially diluted in DMSO and dispensed acoustically to empty wells. Assay media (phenol red-free DMEM (Life Technologies) supplemented with 10% FBS (ATCC)) was dispensed (4 μL/well) and plates shaken at 900 rpm for 10 mins. Cells were seeded at 45,000 cells/well in assay media (4 μL/well), and incubated at 37° C., 5% CO₂ for 1 hour, followed by the addition of IL-2 (R&D Systems; final concentration 300 ng/mL) in pre-warmed assay media (4 μL) for 30 minutes. After cytokine stimulation, cells were lysed with 6 ul of 3× AlphaLisa Lysis Buffer (PerkinElmer) containing 1× PhosStop and Complete tablets (Roche). The lysate was shaken at 900 rpm for 10 minutes at room temperature (RT). Phosphorylated STAT5 was measured via the pSTAT5 AlphaLisa kit (PerkinElmer). Freshly prepared acceptor bead mixture was dispensed onto lysate (5 μL) under green filtered <100 lux light. Plates were shaken at 900 rpm for 2 mins, briefly spun down, and incubated for 2 hrs at RT in the dark. Donor beads were dispensed (5 μL) under green filtered <100 lux light. Plates were shaken at 900 rpm for 2 minutes, briefly spun down, and incubated overnight at RT in the dark Luminescence was measured with excitation at 689 nm and emission at 570 nm using an EnVision plate reader (PerkinElmer) under green filtered <100 lux light.

To determine the inhibitory potency of test compounds in response to IL-2, the average emission intensity of beads bound to pSTAT5 was measured in a human T cell line. IC₅₀ values were determined from analysis of the inhibition curves of signal intensity versus compound concentration. Data are expressed as pIC₅₀ (negative decadic logarithm IC₅₀) values (mean±standard deviation).

### In Vitro Assay Results

TABLE 1

| | JAK1 | JAK2 | JAK3 | Tyk2 | Tall-1 |
|---|---|---|---|---|---|
| Compound | pKᵢ | pKᵢ | pKᵢ | pKᵢ | pIC50 |
| 1 | 10.2 | 10.5 | 10.2 | 9.1 | 8.6 |

Assay 3: Murine (Mouse) Model of IL-13 Induced pSTAT6 Induction in Lung Tissue

IL-13 is an important cytokine underlying the pathophysiology of asthma (Kudlacz et al. Eur. J. Pharmacol, 2008, 582, 154-161). IL-13 binds to cell surface receptors activating members of the Janus family of kinases (JAK) which then phosphorylate STAT6 and subsequently activates further transcription pathways. In the described model, a dose of IL-13 was delivered locally into the lungs of mice to induce the phosphorylation of STAT6 (pSTAT6) which is then measured as the endpoint.

Adult Balb/c mice from Harlan were used in the assay. On the day of study, animals were lightly anesthetized with isoflurane and administered either vehicle or test compound (1 mg/mL, 50 μL total volume over several breaths) via oral aspiration Animals were placed in lateral recumbency post dose and monitored for full recovery from anesthesia before being returned to their home cage. Four hours later, animals were once again briefly anesthetized and challenged with either vehicle or IL-13 (0.03 μg total dose delivered, 50 μL total volume) via oral aspiration before being monitored for recovery from anesthesia and returned to their home cage. One hour after vehicle or IL-13 administration, whole blood and lungs were collected for both pSTAT6 detection in lung homogenates using a Perkin Elmer AlphaLISA® Surefire® Ultra™ HV p-STAT6 (Tyr641) assay kit and for total drug concentration analysis in both lung and plasma. Blood samples were centrifuged (Eppendorf centrifuge, 5804R) for 4 minutes at approximately 12,000 rpm at 4° C. to collect plasma. Lungs were rinsed in DPBS (Dulbecco's Phosphate-Buffered Saline), padded dry, flash frozen, weighed, and homogenized at a dilution of 1:3 in 0.1% formic acid in HPLC water. Plasma and lung levels of test compound were determined by LC-MS analysis against analytical standards constructed into a standard curve in the test matrix. A lung to plasma ratio was determined as the ratio of the lung concentration in ng/g to the plasma concentration in ng/mL at 5 hours.

Activity in the model is evidenced by a decrease in the level of pSTAT6 present in the lungs of treated animals at 5 hours compared to the vehicle treated, IL-13 challenged control animals. The difference between the control animals which were vehicle-treated, IL-13 challenged and the control animals which were vehicle-treated, vehicle challenged dictated the 0% and 100% inhibitory effect, respectively, in any given experiment. The compounds tested in the assay exhibited inhibition of STAT6 phosphorylation at 5 hours after IL-13 challenge as documented below.

TABLE 2
pSTAT6 Inhibition and Plasma/Lung Exposure Observed

| | | | Lung to | pSTAT6 |
|---|---|---|---|---|
| | Lung | Plasma | Plasma | inhibition |
| | Concentration | Concentration | ratio at 5 | at 5 |
| Compound | (ng/g) at 5 hr | (ng/mL) at 5 hr | hr | hours |
| 1 | 10155 ± 1979 | 24.0 ± 16.2 | 423 | 75 |

Observation of significant compound concentration in the mouse lung confirmed that the observed inhibition of IL-13 induced pSTAT6 induction was a result of the activity of the test compound. The lung to plasma ratio at 5 hours showed that compound 1 exhibited significantly more exposure in the lung than exposure in plasma in mice.

Assay 4: Inhibition of TSLP-Evoked TARC Release in Human Peripheral Blood Mononuclear Cells

Thymic stromal lymphopoietin (TSLP) and thymus and activation-regulated chemokine (TARC) are overexpressed in asthmatic airways, and correlate with disease severity. In the lungs, TSLP may be released by bronchial epithelial cells in response to allergens and viral infections. TSLP signals through an IL-7Rα/TSLPR heterodimer found in a broad range of tissues and cell types, including epithelial cells, endothelial cells, neutrophils, macrophages, and mast cells. The binding of TSLP to its receptor induces a conformational change that activates JAK1 and JAK2 to phosphorylate various transcription factors, including STAT3 and STAT5. In immune cells, this triggers a cascade of intracellular events that result in cell proliferation, anti-apoptosis, dendritic cell migration, and production of Th2 cytokines and chemokines. In peripheral blood mononuclear cells (PBMC), TSLP has a proinflammatory effect by activating myeloid dendritic cells to attract and stimulate T cells, a process mediated by the chemoattractant TARC.

In this assay, it was shown that TSLP stimulation induces TARC release from PBMCs, and that this response is attenuated in a dose-dependent manner upon treatment with compound. The potencies of the test compounds were measured for inhibition of TARC release.

PBMC aliquots (previously isolated from whole blood and frozen in aliquots at −80° C.) from 3 to 5 donors were thawed at 37° C. and added dropwise to 40 mL pre-warmed, sterile-filtered, complete RPMI media in 50 mL Falcon tubes. Cells were pelleted and resuspended in complete media at 2.24×10⁶ cells/mL. Cells were seeded at 85 μL (190,000 cells) per well in a tissue culture treated 96-well flat bottom microplate. Cells were allowed to rest for 1 hour at 37° C. with 5% CO₂.

Compounds were received as 10 mM stock solutions in DMSO. 3.7-fold serial dilutions were performed to generate 9 concentrations of test compound in DMSO at 300× the final assay test concentration. 150-fold intermediate dilutions were performed in complete media to generate compound at 2× the final assay test concentration with 0.2% DMSO. After the 1 hour rest period, 95 μL of 2× compound was added to each well of PBMC, for a final assay concentration range of 33.33 μM to 0.95 μM. 95 μL of 0.2% DMSO in complete media was added to the untreated control wells. Cells were pre-treated with compound for 1 hour at 37° C. with 5% CO₂ prior to stimulation.

Recombinant human TSLP protein was reconstituted at 10 μg/mL in sterile DPBS with 0.1% BSA and stored in aliquots at −20° C. Immediately prior to use, an aliquot was thawed and prepared at 20× the final assay concentration in complete media. 10 μL of 20×TSLP was added to each well of PBMC, for a final assay concentration of 10 ng/mL. 10 μL of complete media was added to the unstimulated control wells. Cells were stimulated in the presence of compound for 48 hours at 37° C. with 5% CO₂.

Following stimulation, the cell culture supernatants were harvested and TARC levels were detected by enzyme-linked immunosorbent assay (ELISA), using Human CCL17/TARC Quantikine ELISA Kit (R&D Systems #DDN00) according to the manufacturer's instructions.

For dose response analysis, the log [test compound (M)] was plotted versus the percent response values for each donor, and IC₅₀ values were determined using a nonlinear regression analysis with GraphPad Prism Software using the 4-parameter sigmoidal dose-response algorithm with variable slope. Data are expressed as mean pIC₅₀ (negative decadic logarithm IC₅₀) values calculated from pIC₅₀ values of individual donors and rounded to one decimal place. The potency value for inhibition by compound 1 is summarized in Table 3.

TABLE 3
Potency (pIC₅₀) Values of Compound 1 for Inhibition
of TSLP-evoked TARC Release in Human
Peripheral Blood Mononuclear Cells

| Compound | pIC50 ± st. dev. |
|---|---|
| 1 | 7.2 ± 0.1 |

Assay 5: Pharmacokinetics in Plasma and Lung in Mouse

Plasma and lung concentrations of test compounds and ratios thereof were determined in the following manner BALB/c mice from Charles River Laboratories were used in the assay. Test compounds were individually formulated in 20% propylene glycol in pH 4 citrate buffer at a concentration of 0.2 mg/mL and 50 μL of the dosing solution was introduced into the trachea of a mouse by oral aspiration. At various time points (typically 0.167, 2, 6, 24 hr) post dosing, blood samples were removed via cardiac puncture and intact lungs were excised from the mice. Blood samples were centrifuged (Eppendorf centrifuge, 5804R) for 4 minutes at approximately 12,000 rpm at 4° C. to collect plasma. Lungs were padded dry, weighed, and homogenized at a dilution of 1:3 in sterile water. Plasma and lung concentrations of test compound were determined by LC-MS analysis against analytical standards constructed into a standard curve in the test matrix. A lung-to-plasma ratio was determined as the ratio of the lung AUC in μg hr/g to the plasma AUC in μg hr/mL, where AUC is conventionally defined as the area under the curve of test compound concentration vs. time.

TABLE 4
Plasma and Lung Tissue Exposure Following a Single Oral
Aspiration Administration of Compound 1

| | Plasma | Lung Tissue | Lung Tissue: |
|---|---|---|---|
| | AUC₍₀₋₂₄₎ | AUC₍₀₋₂₄₎ | Plasma |
| Compound | (μg hr/mL) | (μg hr/g) | AUC ratio |
| 1 | 0.943 | 54.5 | 57.8 |

Assay 6: Biochemical ABL1 and ABL2 Kinase Assays

Abl1 and Abl2 assays were performed by measuring the ability of test compounds to compete with enzymatic 33P-ATP incorporation into a peptide substrate. The peptide substrate [EAIYAAPFAKKK] (final concentration 20 μM) was prepared in the reaction buffer (20 mM Hepes (pH 7.5), 10 mM MgCl2, 1 mM EGTA, 0.02% Brij35, 0.02 mg/ml BSA, 0.1 mM Na3VO4, 2 mM DTT) and mixed with recombinant kinase. Serially diluted test compounds (in DMSO; final concentration 1%) were then added and preincubated with the enzyme and substrate mix for 20 minutes at room temperature. ATP was then added to initiate the kinase reactions. The final ATP concentration was 10 μM, and the specific activity of the 33P-ATP was 10 mCi/ml. Kinase reactions were allowed to proceed for 2 hours at room temperature. The reactions were then spotted onto P81 ion exchange paper and the levels of P33 incorporation into the peptide substrate were measured. The kinase activity percent inhibition data were plotted vs. compound concentrations, and IC₅₀ values were determined.

Compound 1 exhibited 90% inhibition at 1 μM in the Abl1 assay and an IC₅₀ value of 15 nM in the Abl2 assay. By comparison, baricitinib exhibited 80% inhibition at 1004 in the Abl1 assay and 35% inhibition at 10 μM in the Abl2 assay.

Clinical Study: A Phase 1, Double-blind, Randomized, Placebo-controlled, Sponsor-open, Single Ascending Dose (SAD) and Multiple Ascending Dose (MAD) Study in Healthy Subjects to Evaluate the Safety, Tolerability, and Pharmacokinetics of Inhaled Compound 1

Study Objectives

The study objectives of Part A were to assess the safety and tolerability of compound 1 following inhaled administration of single ascending doses in healthy subjects, assess the plasma pharmacokinetics (PK) of compound 1 following inhaled administration of single ascending doses in healthy subjects.

The study objectives of Part B were to assess the safety and tolerability of compound 1 following inhaled administration of multiple ascending doses for 7 days in healthy subjects, assess the plasma PK of compound 1 following inhaled administration of multiple ascending doses for 7 days in healthy subjects.

This was a phase 1, 2-part, double-blind, randomized, placebo controlled, sponsor-open, SAD (Part A) and MAD (Part B). Subjects participated in only 1 cohort in only 1 study part.

Part A (SAD) consisted of three (3) cohorts of 8 healthy subjects (6 active and 2 placebo). In each cohort, subjects received a single inhaled dose of compound 1 or placebo. Blood and urine samples were collected for the PK assessment of compound 1 pre-dose and for 72 hours postdose. Cardiodynamic monitoring via Holter monitors was conducted pre-dose and for at least 24 hours following dosing on Day 1 in each cohort, with rest periods for cardiodynamic electrocardiogram (ECG) extractions time matched to the PK sampling time points.

Part B (MAD) consisted of three (3) cohorts of 10 healthy subjects (8 active and 2 placebo). In each cohort, subjects received inhaled doses once daily (QD) for 7 days. Blood samples were collected for the PK assessment of compound 1 pre-dose through 24 hours following the first dose on Day 1 (if QD dosing is used) and pre-dose through 48 hours following dosing on Day 7. Blood samples for PK assessment were also collected pre-dose on the morning of Days 3 through 6. Cardiodynamic monitoring via Holter monitors may be conducted pre-dose on Day 1 and pre-dose and for at least 24 hours following dosing on the morning of Day 7 in each cohort with rest periods for cardiodynamic ECG extractions time matched to the PK sampling time points.

In Parts A and B, safety data (i.e., physical examinations, vital signs, 12-lead safety ECGs, spirometry, clinical laboratory tests, and adverse events [AEs]) were assessed throughout the study; and blood and urine samples were collected for safety assessments. All subjects who received at least one dose of study drug (including subjects who terminated the study early) returned to the CRU 7 (±2) days after the last study drug administration for follow-up procedures, and to determine if any AEs have occurred since the last study visit.

In Part A, subjects in each cohort received a single inhaled dose of compound 1 or placebo using a nebulizer device on Day 1, under fasting conditions.

Doses were as follows:

Cohort A1: 1 mg of compound 1 or matching placebo

Cohort A2: 3 mg of compound 1 or matching placebo

Cohort A3: 10 mg of compound 1 or matching placebo

Hour 0 was defined as the beginning of inhalation in each dose administration.

In Part B, subjects received inhaled doses QD for 7 days using a nebulizer device.

Doses were as follows:

Cohort B1: 1 mg of compound 1 or matching placebo

Cohort B2: 3 mg of compound 1 or matching placebo

Cohort B3: 10 mg of compound 1 or matching placebo

Morning doses on Days 1 and 7 were administered under fasting conditions due to cardiodynamic monitoring. On all other times, doses were administered at least 30 minutes after completion of a meal or snack. Hour 0 was defined as the beginning of inhalation in each morning dose administration.

Results

Compound 1 was well tolerated as single daily doses across a dose range from 1 mg to 10 mg for 7 days in healthy subjects. Adverse events were assessed to be mild or moderate in severity, and none led to discontinuation of study treatment. There were no clinically relevant changes in laboratory parameters, vital signs, or ECGs.

Following single and multiple doses of compound 1, plasma concentrations of compound 1 demonstrated rapid absorption, with a Tₘₐₓ of approximately 1 hour, and a biphasic elimination profile, with a terminal elimination half-life of approximately 24 hours.

TABLE 5
Plasma Pharmacokinetic Parameters (Mean ± SD) Following Inhaled
Administration of Single Ascending Doses of compound 1

| Compound 1 | 1 mg | 3 mg | 10 mg |
|---|---|---|---|
| PK Parameter | (n = 6) | (n = 6) | (n = 6) |
| Cₘₐₓ (ng/mL) | 5.717 (1.6091) | 14.12 (3.3814) | 50.9 (19.272) |
| Tₘₐₓ (hr) | 1.0 (1.0, 1.0) | 1.0 (1.0, 1.0) | 1.0 (1.0, 1.0) |
| t_{1/2} (hr) | 19.29 | 24.73 (2.802) | 23.08 (2.3918) |
| AUC₀₋₂₄ (ng*hr/mL) | 18.51 (7.0649) | 43.33 (14.696) | 152.9 (53.059) |
| AUC_{0-∞} (ng*hr/mL) | 20.71 (8.5482) | 48.58(17.652) | 169.2 (58.374) |

TABLE 6
Plasma Pharmacokinetic Parameters (Mean ± SD) Following Inhaled
Administration of Multiple Ascending Doses of compound 1

| | 1 mg QD | | 3 mg QD | | 10 mg QD | |
|---|---|---|---|---|---|---|
| Compound 1 | (n = 8) | | (n = 8) | | (n = 8) | |
| PK Parameter | Day 1 | Day 7 | Day 1 | Day 7 | Day 1 | Day 7 |
| Cₘₐₓ (ng/mL) | 5.331 | 5.699 | 17.75 | 18.17 | 54.59 | 53.65 |
| | (1.3786) | (1.3426) | (7.4127) | (7.9431) | (27.924) | (21.237) |
| Tₘₐₓ (hr) | 1.0 (1.0, 1.0) | 1.0 (1.0, 1.0) | 1.0 (1.0, 1.0) | 1.0 (1.0, 1.0) | 1.0 (1.0, 1.0) | 1.0 (0.5, 1.0) |
| AUC₀₋₂₄ | 18.42 | 21.57 | 48.15 | 52.3 | 190.9 | 204.4 (105.4) |
| (ng * hr/mL) | (4.6604) | (5.2156) | (17.766) | (19.058) | (117.12) | |

AUC₀₋₂₄, AUC_{0-∞}, and t_{1/2} are presented as arithmetic mean (standard deviation). Tₘₐₓ is presented as median (minimum, maximum).

The value of the binding-corrected JAK IC₅₀ was determined to be 361.6 ng/mL. It was obtained by dividing the JAK IC₅₀ (6.9 ng/mL, obtained from a pIC50 of 7.9 for IL-13-induced STAT6 phosphorylation in the human bronchial epithelial cell line BEAS-2B, based on a MW of 545.7) by the fraction unbound (human plasma protein binding of 98.1%): 6.9 ng/mL/(0.019)=361.6 ng/mL.

The plasma Cₘₐₓ (maximum plasma concentration) values of the compound of formula 1 were found to be well under the binding-corrected JAK IC₅₀, i.e. the plasma concentration necessary to achieve JAK IC₅₀, i.e. the plasma concentration necessary to inhibit Janus kinases by 50%.

The pharmacokinetics of inhaled compound 1 are consistent with low plasma exposures after inhaled administration. Maximal plasma exposures of compound 1 were ˜20-fold and ˜7-fold lower than the protein-adjusted JAK IC₅₀ at dose levels of 3 and 10 mg, respectively.

Absolute NK cell counts were evaluated after multiple-dosing in Part B to assess the potential for systemic pharmacologic effects associated with JAK inhibition by compound 1. No reductions in NK cells were observed relative to baseline in participants receiving placebo or compound 1 at any dose level (1, 3, or 10 mg) explored in the study. The lack of reduction in NK cell counts at any dose level in the study is also consistent with the lack of systemic JAK inhibition; in contrast, marked reductions in NK cell counts have been observed with systemic JAK inhibitors such as tofacitinib (Weinhold, K. J., et al., Reversibility of peripheral blood leukocyte phenotypic and functional changes after exposure to and withdrawal from tofacitinib, a Janus kinase inhibitor, in healthy volunteers. Clin Immunol. 191, 10-20, 2018). Other systemically mediated hematological changes associated with JAK inhibition, including neutrophil and hemoglobin reductions as well as lipid changes, were not observed with inhaled administration of compound 1.

These results support a favorable safety and tolerability profile and PK below levels anticipated to exert systemic effects.

Clinical Study: A Phase 2, Randomized, Double-Blind, Placebo-Controlled, Parallel-Group, Multi-Center Study of Inhaled Compound 1 to Treat Symptomatic Acute Lung Injury Associated with COVID-19

The clinical study was based on a subject population hospitalized with confirmed COVID-19 and requiring supplemental oxygen.

Objectives

In Part 1, the objectives were: to evaluate the safety and tolerability of inhaled compound 1 in subjects with COVID-19, assess the plasma pharmacokinetics (PK) of compound 1 in subjects with COVID-19, characterize the effect of compound 1 on reducing the acute lung injury associated with COVID-19, explore the effect of compound 1 on nasal swab viral load, and blood biomarkers, explore the effect of compound 1 on swab viral infection status, SARS-CoV-2 antibody levels, blood cytokine levels, and biomarkers of inflammation, thrombosis and lung injury.

In Part 2, the primary objective was to characterize the efficacy of compound 1 as measured by respiratory-failure free days (RFDs) through Day 28.

The secondary objectives were to evaluate the effect of compound 1 on: reducing the acute lung injury (as measured by SaO2/FiO2 ratio) associated with COVID-19, safety and tolerability, the clinical outcomes as measured by an 8-point clinical status scale, and the proportion of subjects alive and respiratory failure-free on Day 28. Other objectives included: to characterize the efficacy of compound 1 in reducing the acute lung injury associated with COVID-19, and to characterize the efficacy of compound 1 as measured by ventilator-free days (VFDs), number of days not requiring care in the Intensive Care Unit (ICU-free days), subjects with improvement in oxygenation, dyspnea as measured by the modified Borg Dyspnea Score, the proportion of subjects discharged from hospital during the study, time to hospital discharge, the 28-day mortality rate, and the clinical outcomes as measured by a 6-point clinical status scale.

The exploratory objectives were to evaluate the effect of compound 1 on: time to recovery, the duration and incidence of new oxygen and/or ventilator support, changes in modified HScore, biomarker measures including Severe Acute Respiratory Syndrome-Coronavirus 2 (SARS-CoV-2) viral infection status, SARS-CoV-2 antibodies, blood cytokine levels, and markers indicative of inflammation, thrombosis and lung injury, and population PK.

Other exploratory objectives included: achieving oxygen saturation >90% on room air, changes in chest imaging, changes in fever, biomarker measures including Severe Acute Respiratory Syndrome-associated Coronavirus 2 (SARS-CoV-2) viral load, markers indicative of cytokine storm, SARS-CoV-2 antibodies, and population PK.

Study Design

This was a two-part study. Part 1 was a randomized, double-blind, placebo-controlled, multiple ascending dose study in hospitalized patients with confirmed COVID-19 who require supplemental oxygen. Three ascending-dose cohorts, each comprised of 8 subjects, were dosed at 1 mg, 3 mg and 10 mg single daily dose of compound 1 (except for day 1 where an additional loading dose of 1 mg was given in the 1 mg cohort and an additional loading dose of 3 mg was administered in the 3 mg cohort, there was no loading dose associated with the 10 mg dose), or matched placebo. Six subjects in each cohort were randomized to receive compound 1 and 2 subjects in each cohort were randomized to receive placebo (3:1 randomization). Dosing was once a day. Eligible subjects were randomized and dosed for up to 7 days or until discharge from the hospital, whichever was earlier. At the end of dosing for all subjects in each cohort, the DLRC (Dose Level Review Committee) reviewed unblinded data through Day 7 and results from the same dose level cohort in the corresponding phase 1 study to inform progression to the next dose level and/or to initiate Part 2 of the study. The blinding of subjects' treatment assignments was maintained for off-site personnel who are directly involved with the ongoing operational activities of the study, for all subjects, and for all site personnel until the study was concluded. The activities and composition of DLRC were described in a charter. The DLRC recommended the final doses to carry forward into Part 2. Part 1 assessed safety, tolerability, and PK of compound 1. Serial blood samples were collected from all subjects for PK assessments. Oxygenation data was collected for all subjects, and the ratio of partial pressure of oxygen in arterial blood to the fraction of inspired oxygen (SaO₂/FiO₂) were measured to guide dose selection for Part 2. Subject follow-up after the dosing period was via chart review for 21 days (until Day 28).

Part 2 was a randomized, double-blind, parallel-group study evaluating efficacy and safety of a 3 mg dose of compound 1, with a 6 mg loading dose on day 1, as compared with placebo in hospitalized subjects with confirmed COVID-19 who require supplemental oxygen.

Eligible subjects were stratified by age (≤60 vs >60 years) and concurrent use of antiviral medications (e.g., remdesivir, lopinavir, chloroquine) at baseline. Within each stratum, subjects were randomized 1:1:1 to receive placebo or compound 1. Approximately 20% of participants were enrolled with a baseline clinical status score of six (NIPPV or high flow oxygen device) based on the 8-point ordinal scale (Table 13).

The study drug was administered at 3 mg once-daily in a single dose with an additional 3 mg loading dose on day 1, for up to 7 days or until discharge from the hospital, whichever was earlier. Subjects were followed for up to 28 days or until death, whichever was earlier. Sparse sampling for assessment of compound 1 plasma concentrations was collected for population PK analysis.

Baseline assessments (Day 1) included medical and medication history, vital signs (blood pressure, heart rate, respiratory rate [BP, HR, RR], and body temperature), a physical examination (including height and weight, and hepato- or splenomegaly at a minimum), and measures of oxygenation (arterial blood gas, pulse oximetry, FiO2). Blood samples were collected from all subjects for hematology (complete blood count [CBC] with differential at a minimum), and serum chemistry (renal function, liver function tests, and triglycerides at a minimum). The subject's clinical status was evaluated and inclusion and exclusion criteria were reviewed. Oxygenation was assessed via SaO₂/FiO₂ ratio. Use of a ventilatory and oxygen support, presence in the ICU, clinical status (including mortality), and date of discharge were recorded for all subjects as appropriate. Clinical status was assessed using a 6-point or 8-point scale for all subjects. Changes in nasal swab SARS-CoV-2 viral load, nasal swab SARS-CoV-2 viral infection status, SARS-CoV-2 antibody levels, blood cytokine levels, and blood biomarkers of inflammation, thrombosis, and lung injury were explored.

Subject safety was assessed throughout the study using standard measures, including adverse event (AE) monitoring, physical examinations (including hepato- or splenomegaly at a minimum), vital signs (at a minimum, temperature, BP, HR and respiratory rate (RR)), clinical laboratory tests (at a minimum, CBC with differential, renal function (creatinine, blood urea nitrogen), and liver function tests (aspartate aminotransferase (AST), alanine aminotransferase (ALT), alkaline phosphatase (Alk Phos), and total bilirubin (TBili))), and concomitant medication usage. Subjects were discharged, or considered “ready for discharge” if there was documented evidence of normal body temperature, respiratory rate, and stable oxygen saturation on ambient air or requiring ≤2 L supplemental oxygen.

Duration of Study Participation: 28 days or until death, whichever is earlier.

Number of Subjects Per Group

Part 1 included 24 subjects (8 subjects in each of 3 dose cohorts). Six subjects in each cohort (18 subjects total) received compound 1, and 2 subjects in each cohort (6 subjects total) received placebo.

Part 2 included 210 subjects, including the placebo group.

Compound 1 was administered at 3 mg single daily dose, except for day 1 where an additional 3 mg loading dose was administered (a total of 6 mg was administered on day 1), for up to 7 days, via inhalation using a vibrating mesh nebulizer. Matching placebo was administered once daily for up to 7 days, via inhalation using a vibrating mesh nebulizer.

Study Evaluations

Subjects were assessed daily while hospitalized, with evaluations performed according to the Protocol Schedule of Events. In Part 2, if subjects were discharged from the hospital before Days 14, 21 or 28 and were known to be alive at discharge, they underwent a telephonic study visit on Days 14, 21, and/or 28 to provide data relating to clinical status, adverse events and concomitant medications.

Subject safety was assessed throughout the study using standard measures, including clinical status, AE monitoring, physical examinations, vital signs, clinical laboratory tests and concomitant medication usage.

The following efficacy assessments were conducted: pulse oximetric saturation analyses, use of ventilatory and oxygen support, ICU days, Modified Borg Dyspnea Score, clinical status, modified HScore, and vital signs. The following efficacy assessments were conducted: arterial blood gas analyses, chest imaging (when done for clinical care reasons), and vital signs including temperature.

The following biomarker assessments were conducted: nasal swab for SARS-CoV-2 viral load or infection status, SARS-CoV-2 antibody titers, C-reactive protein (CRP), D-dimer, Fibrinogen and Ferritin, LDH and LDH isoenzymes (Part 2 only), cytokines, and lung injury biomarkers.

Study Endpoints:

Part 1 Endpoints (through Day 7) were: change from baseline in vital signs and clinical laboratory results, incidence and severity of treatment-emergent AEs (TEAEs), pharmacokinetics, Plasma PK parameters on Day 1 and Day 7, Pharmacodynamics (PD), and change from baseline in SaO₂/FiO₂ ratio. Additional Endpoints (through Day 28) were the incidence and severity of TEAEs. Exploratory endpoints included: number of ventilator-free days (VFDs) from randomization to Day 28, number of ICU-free days from randomization to Day 28, area under the curve (AUC) in SaO₂/FiO₂ ratio from Day 1 to Day 7, proportion of subjects with a SaO₂/FiO₂ ratio >300 on Days 5 and 7, proportion of subjects discharged on Days 7, 14, 21 and 28, 28-day all-cause mortality rate, time to hospital discharge, 28 day all-cause mortality rate, change from baseline in the modified Borg Dyspnea Score on Day 7, proportion of subjects in each category of the clinical status scale, as measured with a 6-point ordinal scale on Days 7, 14, 21 and 28, proportion of subjects in each category of vital status (death, discharge, hospitalized) and in the clinical status scale, as measured with an 8-point ordinal scale on Days 7, 14, 21 and 28, proportion of subjects alive and respiratory failure-free on Day 28, proportion of subjects with an oxygen saturation >90% or 93% on room air by study day up to Day 7, proportion of subjects with fever (>37° C. oral or equivalent) by study day up to Day 7, chest imaging (when done for clinical care reasons), modified HScores, biomarkers up to Day 7 (nasal swab for SARS-CoV-2 viral load and infection status, SARS-CoV-2 antibody titers, CRP, D-dimer, fibrinogen, ferritin, lung injury biomarkers, cytokine markers).

In Part 2, the primary endpoint was the number of RFDs from randomization through Day 28. Secondary Endpoints were: change from baseline in SaO₂/FiO₂ ratio on Day 7, proportion of subjects in each category of the 8-point clinical status scale on Days 7, 14, 21 and 28, and proportion of subjects alive and respiratory failure-free on Day 28. The exploratory endpoints were: 28-day all-cause mortality rate, time to recovery (defined as a score of 1, 2, or 3 on the 8-point clinical status scale), duration and incidence of new oxygen use, duration and incidence of new use of ventilator or extracorporeal membrane oxygenation (ECMO), duration and incidence of new non-invasive ventilation or high flow oxygen use, AUC in SaO₂/FiO₂ ratio from Day 1 to Day 7, change from baseline in SaO₂/FiO₂ ratio on Day 5, proportion of subjects with a SaO₂/FiO₂ ratio >315 on Days 5 and 7, proportion of subjects discharged on Days 7, 14, 21 and 28, time to hospital discharge, Change from baseline in the modified Borg Dyspnea Score on Day 7, Number of ICU-free days from randomization through Day 28, Proportion of subjects with an oxygen saturation ≥93% on room air, Modified HScores, Biomarkers (including Nasal SARS-CoV-2 viral infection status, SARS-CoV-2 antibody titers, CRP (standard or high sensitivity), D-dimer, fibrinogen, ferritin, LDH and LDH isoenzymes, Cytokines, Lung injury biomarkers). Additional exploratory endpoints may include: change from baseline in SaO₂/FiO₂ ratio in mmHg on Day 7, number of VFDs from randomization to Day 28, proportion of subjects with a SaO₂/FiO₂ ratio >300 on Days 5 and 7, proportion of subjects in each category of the 6-point clinical status scale on Days 7, 14, 21 and 28, proportion of subjects in each category of vital status (death, discharge, hospitalized) on Days 7, 14, 21 and 28, proportion of subjects with an oxygen saturation >90% on room air, proportion of subjects with fever (>37° C. oral or equivalent), chest imaging (when done for clinical care reasons), biomarkers including: nasal SARS-CoV-2 viral load. The safety endpoints were: change from baseline in vital signs and clinical laboratory results, and incidence and severity of TEAEs. The PK endpoints were population PK parameters for compound 1.

Analysis of Part 2:

The primary endpoint was the number of respiratory failure-free days (RFDs) from randomization through Day 28. A RFD was defined as a day that a subject is alive and not requiring the use of invasive mechanical ventilation, non-invasive positive pressure ventilation, high-flow oxygen devices, or oxygen supplementation from randomization through Day 28. A clinical status score of <4 (Table 13) was equivalent to a respiratory failure-free day. The number of RFDs was 0 for subjects who use respiratory support for 28 days or longer, or for subjects who died on or before Day 28.

Treatment comparisons were performed using a Van Elteren test (a stratified Wilcoxon rank sum test) adjusting for stratification factors. Treatment difference were summarized based on median of RFD between compound 1 and placebo. Additional prognostic baseline covariates (e.g., comorbidities) were included in the sensitivity analyses.

For SaO₂/FiO₂, treatment comparisons versus placebo were conducted using a mixed-model repeated measures (MMRM) model. The model included fixed effects for randomized treatment group, study day (Day 7, 14, 21 and 28), treatment group by study day interaction, baseline SaO₂/FiO₂ ratio, treatment group by baseline SaO₂/FiO₂ ratio interaction, and stratification factors (baseline age group ≤60 vs >60 years, and concurrent use of antiviral medication at baseline Yes vs. No). A random effect for subject was also included in the model. An unstructured covariance matrix was used to estimate covariance of within-patient scores. The Kenward-Roger approximation was used to estimate denominator degrees of freedom. From this model, least squares means, standard errors, treatment differences in LS means, and 95% confidence intervals (CIs) were estimated. Each dose of compound 1 was compared against placebo and 2-sided nominal p-value were reported.

Another endpoint was the number of ventilator-free days (VFDs) from randomization to Day 28. A VFD was defined as a day that a subject is alive and successfully not using invasive mechanical ventilation or non-invasive positive pressure ventilation from randomization to Day 28. The number of VFDs was 0 for subjects who use ventilatory support for 28 days or longer, or for subjects who die on or before Day 28. Treatment comparisons were performed using a Van Elteren test (a stratified Wilcoxon rank sum test) adjusting for stratification factors. Treatment difference were summarized based on median of VFD between compound 1 and placebo.

Results from Part 1

Compound 1 was generally well tolerated as a single daily dose of 1 mg, 3 mg, and 10 mg administered for 7 consecutive days in patients with COVID-19. The DLRC determined that it was safe to proceed to Part 2 with a single daily dose of 3 mg, and/or a single daily dose of 10 mg in patients with COVID-19. A 3 mg single daily dose of compound 1 with a 6 mg loading dose on day 1 was selected for Part 2. The 6 mg loading dose on Day 1 was selected to achieve steady-state of compound 1 concentrations in the lung after the initial dose. Selection of the 6 mg loading dose (a 2-fold increase over the 3 mg maintenance dose) was based on the observed terminal elimination half-life (24.7 hr at 3 mg) in human plasma determined in the Phase 1 study in healthy volunteers. Based on pharmacokinetic modeling and the assumption of similar elimination rates in human lung tissue and plasma after inhalation dosing, a two-fold higher loading dose was anticipated to result in rapid attainment of the steady-state exposure on Day 1 for a 3 mg once-daily dosing regimen.

The rationale for the Day 1 loading dose was to provide an immediate, high level of estimated target attainment by reaching target levels in the lung on Day 1 that approximate those that otherwise would be reached by once a day dosing several days later at steady state. The goal of early target attainment was to reach effective immunosuppression levels fast. Based on the potential rapid course of the acute lung injury resulting from COVID-19, the dosing schedule was designed to arrest the over-release of damaging cytokines and shut off the over-active inflammatory response quickly.

Compound 1 demonstrated low plasma exposure relative to protein-adjusted IC₅₀ for JAK inhibition based on BEAS-2B data. Compound 1's PK was similar in COVID-19 patients compared to healthy volunteers. The loading dose on Day 1 provided exposures consistent with pseudo steady-state.

In part 1, the majority of subjects received glucocorticoids (dexamethasone) and anticoagulation (heparin). More specifically, 83.3% of the placebo, 1 mg, and 10 mg groups received dexamethasone while all 3 mg group subjects received dexamethasone. Three subjects received remdesivir, one in the placebo, one in the 3 mg, and one in the 10 mg group. The majority of subjects had hypertension, diabetes, and sleep apnea.

TABLE 7
Mortality, Proportion of Respiratory Failure-free and Time to Hospital
Discharge Data

| | | Compound 1 | Compound 1 | Compound 1 |
|---|---|---|---|---|
| | Placebo | 1 mg | 3 mg | 10 mg |
| | (N = 6) | (N = 6) | (N = 7) | (N = 6) |
| N | 6 | 6 | 7* | 6 |
| # Subjects | 4 (66.7%) | 5 (83.3%) | 7 (100.0%)* | 6 (100.0%) |
| Alive | | | | |
| # Subjects | 4 (66.7%) | 5 (83.3%) | 6 (85.7%)* | 6 (100.0%) |
| Respiratory | | | | |
| Failure-Free | | | | |
| Proportion | 4 (66.7%) | 5 (83.3%) | 6 (85.7%)* | 6 (100.0%) |
| of Subjects | | | | |
| Alive and | | | | |
| Respiratory | | | | |
| Failure-free | | | | |
| Died (All- | 2 (33.3%) | 1 (16.7%) | 0 | 0 |
| Cause | | | | |
| Mortality) | | | | |
| Time to | 22.50 ± 6.442 | 18.83 ± 6.795 | 15.29 ± 6.651 | 15.17 ± 4.446 |
| Hospital | | | | |
| Discharge | | | | |
| (Days) | | | | |
| Mean ± SD | | | | |
| Median | 24.5 | 18.5 | 17.0 | 16.50 |

*One subject discontinued the study due to negative SARS-COV-2 test and was discharged alive but lost to follow-up. This subject was counted as respiratory failure.
Note:
subjects who were still hospitalized or died prior to study Day 28 were assigned the worst outcome (a time to hospital discharge of 28 days).

TABLE 8
Modified Borg Dyspnea Scores Change from Baseline

| | | | Compound 1 | Compound 1 | Compound 1 |
|---|---|---|---|---|---|
| | | Placebo | 1 mg | 3 mg | 10 mg |
| | | (N = 6) | (N = 6) | (N = 7) | (N = 6) |
| Baseline | n | 6 | 6 | 7 | 6 |
| | Mean (SD) | 5.67 | 6.17 | 6.71 | 6.00 |
| | | (2.503) | (0.408) | (0.951) | (2.966) |
| Day 2 | n | 6 | 6 | 7 | 6 |
| | Mean (SD) | −0.17 | 0.33 | −0.43 | −0.33 |
| | | (0.983) | (0.516) | (0.976) | (1.033) |
| Day 3 | n | 5 | 6 | 7 | 6 |
| | Mean (SD) | 0.20 | −0.33 | −1.00 | −1.17 |
| | | (1.304) | (0.816) | (1.000) | (1.169) |
| Day 4 | n | 5 | 6 | 6 | 6 |
| | Mean (SD) | 0.60 | −0.83 | −0.50 | −1.50 |
| | | (2.408) | (0.408) | (0.548) | (1.871) |
| Day 5 | n | 4 | 6 | 5 | 6 |
| | Mean (SD) | 0.25 | −1.17 | −1.20 | −1.17 |
| | | (2.217) | (0.753) | (0.837) | (2.563) |
| Day 6 | n | 3 | 6 | 5 | 6 |
| | Mean (SD) | −0.33 | −1.33 | −1.20 | −1.33 |
| | | (1.155) | (0.816) | (0.837) | (2.422) |
| Day 7 | n | 3 | 6 | 5 | 6 |
| | Mean (SD) | −0.33 | −1.50 | −1.80 | −1.67 |
| | | (1.155) | (1.049) | (0.837) | (2.658) |

TABLE 9
SaO₂/FiO₂ Ratio Change from Baseline at Day 7

| | | Compound 1 | Compound 1 | Compound 1 |
|---|---|---|---|---|
| | Placebo | 1 mg | 3 mg | 10 mg |
| Value | (N = 6) | (N = 6) | (N = 7) | (N = 6) |
| n | 6 | 6 | 5 | 5 |
| Mean (SD) | 235.04 (122.692) | 403.92 (96.468) | 360.53 (93.042) | 303.61 (87.108) |
| Median | 198.65 | 453.81 | 348.15 | 276.47 |
| Q1,Q3 | 146.15, 260.86 | 328.67, 472.86 | 273.53, 447.62 | 245.00, 304.19 |
| Min, Max | 144.0, 461.9 | 241.5, 472.9 | 268.6, 464.8 | 240.0, 452.4 |
| Change from | | | | |
| Baseline | | | | |
| n | 6 | 6 | 5 | 5 |
| Mean (SD) | −49.50 (65.336) | 108.91 (87.914) | 106.38(87.780) | 11.23 (106.340) |
| Median | −69.22 | 131.62 | 121.24 | 2.18 |
| Q1, Q3 | −86.29, −6.57 | 58.10, 175.24 | 46.70, 173.21 | −53.75, 31.05 |
| Min, Max | −123.4, 57.7 | −40.9, 197.7 | −10.3, 201.0 | −101.4, 178.1 |

TABLE 10
Clinical Status at Day 28

| | | Compound 1 | Compound 1 | Compound 1 |
|---|---|---|---|---|
| | Placebo | 1 mg | 3 mg | 10 mg |
| | (N = 6) | (N = 6) | (N = 7) | (N = 6) |
| N | 6 | 6 | 6 | 6 |
| 1-Not hospitalized, no limitations on | 3 (50.0%) | 5 (83.3%) | 5 (83.3%) | 5 (83.3%) |
| activities | | | | |
| 2-Not hospitalized, but with | 0 | 0 | 0 | 1 (16.7%) |
| limitations on activities and/or | | | | |
| requiring home oxygen | | | | |
| 3-Hospitalized, not requiring | 0 | 0 | 0 | 0 |
| supplemental oxygen, and no longer | | | | |
| requiring ongoing medical care | | | | |
| 4-Hospitalized, not requiring | 1 (16.7%) | 0 | 1 (16.7%) | 0 |
| supplemental oxygen, but requiring | | | | |
| ongoing medical care (whether or | | | | |
| not related to CO VID-19) | | | | |
| 5-Hospitalized, requiring | 0 | 0 | 0 | 0 |
| supplemental oxygen | | | | |
| 6-Hospitalized, on non-invasive | 0 | 0 | 0 | 0 |
| ventilation or high-flow oxygen | | | | |
| devices | | | | |
| 7-Hospitalized, on invasive | 0 | 0 | 0 | 0 |
| mechanical ventilation or | | | | |
| extracorporeal membrane | | | | |
| oxygenation | | | | |
| 8-Death | 2 (33.3%) | 1 (16.7%) | 0 | 0 |

TABLE 11
Hospital Discharges at Days 7, 14, 21 and 28

| | | Compound 1 | Compound 1 | Compound 1 |
|---|---|---|---|---|
| | Placebo | 1 mg | 3 mg | 10 mg |
| | (N = 6) | (N = 6) | (N = 7) | (N = 6) |
| Hospital | | | | |
| Discharge | | | | |
| Day 7 | 0 | 0 | 1 (14.3%) | 0 |
| Day 14 | 1 (16.7%) | 2 (33.3%) | 2 (28.6%) | 2 (33.3%) |
| Day 21 | 3 (50.0%) | 3 (50.0%) | 6 (85.7%) | 6 (100%) |
| Day 28 | 3 (50.0%) | 5 (83.3%) | 7 (100%) | 6 (100%) |
| Still in | 1 (16.7%) | 0 | 0 | 0 |
| Hospital at | | | | |
| Day 28 | | | | |

All of the compound 1 groups demonstrated a positive trend when compared to Placebo in:
improved oxygenation (SaO₂/FiO₂ Ratio) in mean change from baseline to Day 7 whereas placebo subjects trended downward;
clinical improvement as measured by the 8-point Clinical Status Scale from Day 1 to Day 28 (trend in improvement seen on day 7, 14, 21, and 28);
% subjects alive/reduced mortality;
% subjects respiratory-failure free at Day 28;
earlier time to hospital discharge; and
improvement in Modified Borg Dyspnea Score in mean change from baseline at Day 7 (Rubina M. Khair et al., The Minimal Important Difference in Borg Dyspnea Score in Pulmonary Arterial Hypertension. Ann. Am. Thorac. Soc., 2016 June; 13(6): 842-849).

By Day 21, 86% and 100% of subjects in the 3 mg and 10 mg treatment groups, respectively, were discharged from the hospital, as compared to 50% in the Placebo group.

There were three deaths observed: two in the placebo group and one in the 1 mg group.

TABLE 12
Inflammation and Epithelial Injury Biomarkers (within-group percent difference
in geometric mean from baseline and corresponding 95% confidence interval)

| | Placebo | Compound 1 | Compound 1 | Compound 1 |
|---|---|---|---|---|
| Biomarker | (N = 6) | 1 mg (N = 6) | 3 mg (N = 7) | 10 mg (N = 6) |
| hsCRP | 41.01 | −34.85 | −74.95 | −52.13 |
| | (−59.31,388.70) | (−94.58, 682.48) | (−92.59, −15.35) | (−93.66, 261.67) |
| IFN-γ | −68.57 | −46.12 | −90.42 | −78.59 |
| | (−97.26, 261.14) | (−97.29, 972.89) | (−99.41, 55.11) | (−98.90,316.31) |
| IP-10 | −59.60 | −20.65 | −81.44 | −79.32 |
| | (−84.95, 8.50) | (−90.46, 559.78) | (−97.88, 62.53) | (−93.20, −37.05) |
| IL-6 | −35.51 | 29.45 | −80.24 | −58.84 |
| | (−77.32, 83.43) | (−46.25, 211.76) | (−96.94, 27.39) | (−94.24, 194.02) |
| MCP-1 | −31.71 | 13.90 | −54.15 | −21.15 |
| | (−78.85, 120.49) | (−35.57, 101.35) | (−69.79, −30.40) | (−62.89, 67.53) |
| MDC | −15.65 | −55.03 | −23.84 | −6.70 |
| | (−44.17, 27.43) | (−71.10, −30.02) | (−62.90, 56.35) | (−33.89, 31.67) |
| TARC | −7.12 | −57.21 | −34.57 | −21.21 |
| | (−47.30, 63.71) | (−76.45, −22.24) | (−69.33, 39.60) | (−45.76, 14.45) |
| IL-10 | −23.98 | −26.59 | −70.10 | −40.57 |
| | (−80.03, 189.44) | (−83.20, 220.70) | (−87.27, −29.80) | (−91.42, 311.70) |
| IL-8 | −16.24 | −16.78 | −48.02 | −25.87 |
| | (−79.14, 236.28) | (−71.69, 144.59) | (−80.68, 39.85) | (−56.04, 25.02) |
| RAGE | −36.90 | −14.22 | −82.95 | −54.44 |
| | (−76.44, 69.00) | (−71.98, 162.61) | (−92.47, −61.38) | (−78.45, −3.70) |

For hsCRP, n=5 in each of placebo and compound 1 groups (n is the number of subjects with matched D1 and D7 samples). For the other biomarkers, n=6, 6, 4 and 6 in placebo, and compound 1 1 mg, 3 mg and 10 mg groups, respectively.

Compound 1 demonstrated a positive trend mostly in the 3 mg and 10 mg doses in:
reduction of inflammation markers including hsCRP, IFN-g, IL-6, IP-10 and
reduction in alveolar epithelial cell injury marker RAGE.

RAGE and PSP-D are associated with respiratory airway distress syndrome as biomarkers for epithelial damage.

TABLE 13
Clinical Status Score

| Status/Criteria | Score |
|---|---|
| Death | 8 |
| Hospitalized, on invasive mechanical ventilation or extracorporeal membrane | 7 |
| oxygenation | |
| Hospitalized, on non-invasive ventilation or high-flow oxygen devices | 6 |
| Hospitalized, requiring supplemental oxygen | 5 |
| Hospitalized, not requiring supplemental oxygen, but requiring ongoing medical | 4 |
| care (whether or not related to COVID-19) | |
| Hospitalized, not requiring supplemental oxygen, and no longer requiring ongoing | 3 |
| medical care (including subjects hospitalized for infection control) | |
| Not hospitalized, but with limitation on activities and/or requiring home oxygen | 2 |
| Not hospitalized, no limitations on activities | 1 |

High-flow devices include high-flow nasal cannula (heated, humidified, oxygen delivered via reinforced nasal cannula at flow rates >20 L/min with fraction of delivered oxygen ≥0.5).

Results from Part 2

Efficacy outcomes, n=210 Intent-to-treat (ITT) population
Primary: no statistically significant difference in RFDs from randomization through Day 28 between compound 1 and placebo in ITT (median: 21 vs. 21 days; p=0.61).
Secondary: no difference in change from baseline at Day 7 in SaO₂/FiO₂ ratio, proportion of patients in each category of the 8-point Clinical Status scale, and proportion of patients alive and respiratory failure-free at Day 28.

Compound 1 demonstrated a favorable trend in improvement when compared to placebo for 28-day all-cause mortality (total number of deaths: 6 vs. 13, HR: 0.42, p=0.08) and time to recovery (median: 10 vs. 11 days, HR: 1.27, p=0.12).
In a post-hoc analysis of patients with baseline CRP (n=201):
In patients with CRP <150 mg/L (n=171), there was an improvement in those treated with compound 1 when compared to placebo in:
28-day all-cause mortality (total number of deaths: 1 vs 9, HR: 0.097, p=0.009) and
time to recovery (median: 10 vs. 11 days, HR: 1.48, p=0.02).
In patients with CRP >150 mg/L (n=30), there was no difference in time to recovery or 28-day all-cause mortality between those treated with compound 1 or placebo.

### Safety:

Compound 1 was well-tolerated; adverse events and serious adverse events occurred in 34.0% and 9.7% of patients treated with compound 1, and 41.2% and 15.7% of patients treated with placebo, respectively.
Adverse events of liver abnormalities or disease occurred in 9.7% and 7.8% of patients treated with compound 1 and placebo, respectively.
Serious infections and venous thromboembolism occurred in 1.0% and none of the patients treated with compound 1, and 2.0% and 4.9% in patients treated with placebo, respectively.

Plasma exposure of compound 1 was low and consistent with expectations for a lung-selective medicine.

TABLE 14
Summary of Patient Population

| | Compound 1 | Placebo | Total |
|---|---|---|---|
| n (%) | n = 106 | n = 104 | n = 210 |
| Patients randomized | 103 (100%) | 102 (100%) | 205 (100%) |
| and treated with study | | | |
| drug | | | |
| Patients completed | 92 (89.3%) | 89 (87.3%) | 181 (88.3%) |
| study | | | |
| Patients discontinued | 11 (10.7%) | 13 (12.7%) | 24 (11.7%) |
| from study | | | |
| Reasons for withdrawal | | | |
| Adverse event | 8 (7.8%) | 13 (12.7%) | 21 (10.2%) |
| Lost to follow-up | 1 (1.0%) | 0 | 1 (0.5%) |
| Withdrawal by | 2 (1.9%) | 0 | 2 (1.0%) |
| patients | | | |

24 patients discontinued early from the study: 21 for AEs (19 leading to death), 2 by patient's choice, and 1 was lost to follow-up after being discharged to a different hospital.

TABLE 15
Baseline demographics and clinical characteristics

| | Compound l n = 106 | Placebo n = 104 |
|---|---|---|
| Mean age, years ± SD | 58.3 ± 12.42 | 58.1 ± 12.54 |
| Male, n (%) | 65 (61.3%) | 63 (60.6%) |
| White, n (%) | 104 (98.1%) | 102 (98.1%) |
| Mean BMI, kg/m² ± SD | 30.10 ± 3.71 | 30.10 ± 4.12 |
| Number of comorbidities, | | |
| % | | |
| 1 | 23.6% | 24.0% |
| ≥2 | 46.2% | 45.2% |
| Overall corticosteroids, % | 98.1% | 100% |
| Dexamethasone, % | 91.3% | 91.2% |
| Remdesivir, n (%) | 12 (11.7%) | 7 (6.9%) |
| Mean oxygen, L/min ± SD | 7.33 ± 7.24 | 6.73 ± 2.73 |
| Mean CRP, mg/L ± SD | 75.26 ± 72.21 | 70.54 ± 70.13 |
| CS 5: n = 169 (80%)* | n = 87 | n = 82 |
| CS 6: n = 39 (19%)* | n = 18 | n = 21 |

*2 patients without baseline Clinical Status Score (CS)

While the present disclosure has been described with reference to specific aspects or embodiments thereof, it will be understood by those of ordinary skilled in the art that various changes can be made or equivalents can be substituted without departing from the true spirit and scope of the disclosure. Additionally, to the extent permitted by applicable patent statutes and regulations, all publications, patents and patent applications cited herein are hereby incorporated by reference in their entirety to the same extent as if each document had been individually incorporated by reference herein.

1. A method of treating a patient infected with a coronavirus and having a baseline level of CRP below 150 mg/L, comprising administering to the patient a compound of formula 1: 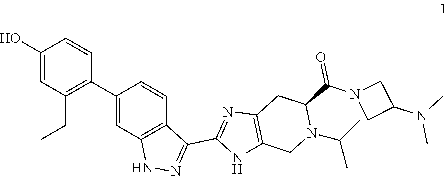 [SMILES: CCc1cc(O)ccc1-c1ccc2c(-c3nc4c([nH]3)CN(C(C)C)[C@H](C(=O)N3CC(N(C)C)C3)C4)n[nH]c2c1]
or a pharmaceutically-acceptable salt thereof.

2. The method of claim 1, wherein the coronavirus is selected from the group consisting of SARS-CoV-1, SARS-CoV-2, and MERS-CoV.

3. The method of claim 1, wherein the coronavirus is SARS-CoV-2.

4. The method of claim 1, wherein the compound, or a pharmaceutically-acceptable salt thereof, is administered by inhalation.

5. The method of claim 1, wherein the compound, or a pharmaceutically-acceptable salt thereof, is administered by nebulized inhalation.

6.-10. (canceled)

11. The method of claim 1, wherein the compound, or a pharmaceutically-acceptable salt thereof, is administered once a day.

12. The method of claim 1, wherein the compound, or a pharmaceutically-acceptable salt thereof, is administered at a higher loading dose on day 1 of administration followed by a lower dose on the following days.

13.-21. (canceled)

22. The method of claim 1, wherein the method comprises administering one or more additional therapeutic agents or treatments to the patient.

23. The method of claim 22, wherein the one or more additional therapeutic agents are selected from the group consisting of: an IL-6 inhibitor, an IL-6 receptor antagonist, an IL-6 receptor agonist, an IL-2 inhibitor, an antiviral, an anti-inflammatory drug, a sodium-glucose cotransporter 2 inhibitor, a vaccine, an ACE2 inhibitor, an antibiotic, an antiparasitic, a sphingosine 1-phosphate receptor modulator, a TMPRSS2 inhibitor, a TNF alpha inhibitor, an anti-TNF, a membrane haemagglutinin fusion inhibitor, an inhibitor of the terminal glycosylation of ACE2, a CCR5 inhibitor, stem cells, allogeneic mesenchymal stem cells, CRISPR therapy, CAR-T therapy, TCR-T therapy, a virus-neutralizing monoclonal antibody, a protease inhibitor, a SARS-CoV-2 antibody, a siRNA, a plasma-derived immunoglobulin therapy, a S-protein modulator, a PLX stem cell therapy, chimeric humanized virus suppressing factor, multipotent adult progenitor cell therapy, an anti-viroporin, umbilical cord-derived mesenchymal stem cells, a polymerase inhibitor, autologous adipose-derived mesenchymal stem cells, an angiotensin converting enzyme 2 inhibitor, an immunoglobulin agonist, a nucleoside reverse transcriptase inhibitor, a cytotoxic T-lymphocyte protein-4 inhibitor, a lung surfactant associated protein D modulator, a protease inhibitor, a nuclear factor kappa B inhibitor, a xanthine oxidase inhibitor, an endoplasmin modulator, a CCL26 gene inhibitor, a TLR modulator, a TLR agonist, a TLR-2 agonist, a TLR-6 agonist, a TLR-9 agonist, a TLR-4 agonist, a TLR-7 agonist, a TLR-3 agonist, an opioid receptor antagonist, a moesin inhibitor, an angiotensin converting enzyme 2 modulator, a MEK protein kinase inhibitor, aCD40 ligand receptor agonist, a CD70 antigen modulator, an amyloid protein deposition inhibitor, an apolipoprotein gene stimulator, a bromodomain containing protein 2 inhibitor, a bromodomain containing protein 4 inhibitor, an IL-15 receptor agonist, an immunoglobulin gamma Fc receptor III agonist, a MEK-1 protein kinase inhibitor, a Ras gene inhibitor, an interferon beta ligand, a galectin-3 inhibitor, a heat shock protein inhibitor, an elongation factor 1 alpha 2 modulator, a VEGF-1 receptor modulator, an Angiotensin II AT-2 receptor agonist, a basigin inhibitor, a viral envelope glycoprotein inhibitor, a gelsolin stimulator, a trypsin inhibitor, a GM-CSF ligand inhibitor, a urokinase plasminogen activator inhibitor, a serine protease inhibitor, a PDE 3 inhibitor, a PDE 4 inhibitor, a C-reactive protein inhibitor, a chemokine CC22 ligand inhibitor, a GM-CSF receptor antagonist, an hemoglobin scavenger receptor antagonist, a metalloprotease-1 inhibitor, a metalloprotease-3 inhibitor, a metalloprotease inhibitor, a small inducible cytokine A17 ligand inhibitor, a VEGF gene inhibitor, a Coronavirus spike glycoprotein inhibitor, a nucleoprotein inhibitor, an ATP binding cassette transporter B5 modulator, a vimentin modulator, a stem cell antigen-1 inhibitor, a casein kinase II inhibitor, a complement C5a factor inhibitor, an aldose reductase inhibitor, a calpain-I inhibitor, a calpain-II inhibitor, a calpain-IX inhibitor, a proto-oncogene Mas agonist, a non-nucleoside reverse transcriptase inhibitor, an Interferon gamma ligand inhibitor, a CD4 modulator, a TGFB2 gene inhibitor, an Interleukin-1 beta ligand inhibitor, an inosine monophosphate dehydrogenase inhibitor, an angiotensin converting enzyme 2 stimulator, an adenosine A3 receptor agonist, a palmitoyl protein thioesterase 1 inhibitor, a Btk tyrosine kinase inhibitor, a NK1 receptor antagonist, an acetaldehyde dehydrogenase inhibitor, a CGRP receptor antagonist, a prostaglandin E synthase-1 inhibitor, a VIP receptor agonist, a nuclear factor kappa B gene modulator, a Grp78 calcium binding protein inhibitor, a Jun N terminal kinase inhibitor, a transferrin modulator, a p38 MAP kinase modulator, a CCR5 chemokine antagonist, a APOA1 gene stimulator, a bromodomain containing protein 2 inhibitor, a bromodomain containing protein 4 inhibitor, a BMP10 gene inhibitor, a BMP15 gene inhibitor, an adrenergic receptor antagonist, a human papillomavirus E6 protein modulator, a human papillomavirus E7 protein modulator, a Ca2+ release activated Ca2+ channel 1 inhibitor, an amyloid protein deposition inhibitor, a gamma-secretase inhibitor, a 2,5-Oligoadenylate synthetase stimulator, an Interferon type I receptor agonist, a ribonuclease stimulator, a S phase kinase associated protein 2 inhibitor, a dehydropeptidase-1 modulator, a calcium channel modulator, a signal transducer CD24 modulator, a cyclin E inhibitor, a cyclin-dependent kinase-2 inhibitor, a cyclin-dependent kinase-5 inhibitor, a cyclin-dependent kinase-9 inhibitor, a GM-CSF ligand inhibitor, an Interferon receptor modulator, an Interleukin-29 ligand, a cyclin-dependent kinase-7 inhibitor, a MCL1 gene inhibitor, a complement C5 factor inhibitor, an heparin agonist, an exo-alpha sialidase modulator, a muscarinic receptor antagonist, an IL-8 receptor antagonist, a vitamin D3 receptor agonist, a high mobility group protein B1 inhibitor, a CASP8-FADD-like regulator inhibitor, an ecto NOX disulfide thiol exchanger 2 inhibitor, a sphingosine kinase inhibitor, a sphingosine-1-phosphate receptor-1 antagonist, a stimulator of interferon genes protein stimulator, a topoisomerase inhibitor, an X-linked inhibitor of apoptosis protein inhibitor, an angiopoietin ligand-2 inhibitor, a neuropilin 2 inhibitor, a listeriolysin stimulator, an Interferon gamma receptor agonist, a MAPK gene modulator, a GM-CSF ligand inhibitor, an immunoglobulin G1 modulator, an immunoglobulin kappa modulator, a kallikrein modulator, a mannan-binding lectin serine protease inhibitor, an ubiquitin modulator, an IL12 gene stimulator, a xanthine oxidase inhibitor, a dihydroorotate dehydrogenase inhibitor, an IL-17 antagonist, a MAP kinase inhibitor, a PARP inhibitor, a poly ADP ribose polymerase 1 inhibitor, a poly ADP ribose polymerase 2 inhibitor, a dipeptidyl peptidase I inhibitor, a Btk tyrosine kinase inhibitor, a type I IL-1 receptor antagonist, an exportin 1 inhibitor, a hyaluronidase inhibitor, a sodium glucose transporter-2 inhibitor, a dihydroceramide delta 4 desaturase inhibitor, a sphingosine kinase 2 inhibitor, an Interferon beta ligand, an ICAM-1 stimulator, a TNF antagonist, a vascular cell adhesion protein 1 agonist, a COVID19 Spike glycoprotein modulator, a complement C1s subcomponent inhibitor, a NMDA receptor epsilon 2 subunit inhibitor, a tankyrase-1 inhibitor, a protein translation initiation inhibitor, a sigma receptor modulator, a sigmaR1 receptor modulator, a sigmaR2 receptor modulator, an antihistamine, an anti-C5aR, a RNAi. a corticosteroid, a BCR-ABL a tyrosine kinase inhibitor, a colony stimulating factor, an inhibitor of tissue factor (TF), a recombinant granulocyte macrophage colony-stimulating factor (GM-CSF), a Gardos channel blocker, a heat-shock protein 90 (Hsp90) inhibitor, an alpha blocker, a cap binding complex modulator, a LSD1 inhibitor, a CRAC channel inhibitor, a RNA polymerase inhibitor, a CCR2 antagonist, a DHODH inhibitor, a blood thinner, an anti-coagulant, a factor Xa inhibitor, a SSRI, a SNRI, a sigma-1 receptor activator, a beta-blocker, a caspase inhibitor, a serine protease inhibitor, an IL-23A modulator, a NLRP3 inhibitor, an Angiopoietin-Tie2 signaling pathway modulator, a mannan-binding lectin-associated serine protease-2 modulator, a PDE4 inhibitor, a Vasoactive Intestinal Polypeptide, a microtubule depolymerization agent, a (PD)-1 checkpoint inhibitor, an Axl kinase inhibitor, a (PD)-1/PD-L1 checkpoint inhibitor, a PD-L1 checkpoint inhibitor, a T-cell CD61 receptor modulator, a Factor XIIa antagonist, an oral spleen tyrosine kinase (SYK) inhibitor, a CK2 inhibitor, a NMDA receptor antagonist, a SK2 inhibitor, an antiandrogen and a tankyrase-2 inhibitor.

24. The method of claim 22, wherein the one or more additional therapeutic agents are selected from the group consisting of: cidofovir triphosphate, cidofovir, abacavir, ganciclovir, stavudine triphosphate, 2′-O-methylated UTP, desidustat, ampion, trans sodium crocetinate, CT-P59, Ab8, heparin, Apixaban, GC373, GC376, Oleandrin, GS-441524, sertraline, Lanadelumab, zilucoplan, abatacept, CLBS119, Ranitidine, Risankizumab, AR-711, AR-701, MP0423, bempegaldesleukin, melatonin, carvedilol, mercaptopurine, paroxetine, casirivimab, imdevimab, ADG20, emricasan, dapansutrile, ceniciviroc infliximab, DWRX2003, AZD7442, MAN-19, LAU-7b, niclosamide, ANA001, fluvoxamine, narsoplimab, Sarconeos, GIGA-2050, VERU-111, REGN-COV2, icatibant, cenicriviroc, NTR-441, LAM-002A, oseltamivir, VHH72-Fc, MK-4482, EB05, OB-002, CM-4620-IE, IMU-838, SNG001, NT-17, BOLD-100, WP1122, itolizumab, PB1046, fostamatinib, colchicine, M5049, EDP1815, ABX464, CPI-006, azelastine, garadacimab, silmitasertib, lopinavir, ritonavir, remdesivir, cloroquine, hydrochloroquine, convalescent plasma transfusion, azithromycin, tocilizumab, famotidine, sarilumab, interferon beta, interferon beta-1a, interferon beta-1b, peginterferon lambda-1a, favipiravir, ASDC-09, dapagliflozin, CD24Fc, ribavirin, umifenovir, nitric oxide, APN01, teicoplanin, oritavancin, dalbavancin, monensin, ivermectin, darunavir, cobicistat, fingolimod, camostat, galidesicir, thalomide, leronlimab, remestemcel-L, canakinumab, TAK-888, azvudine, BPI-002, AT-100, T-89, Neumifil, GreMERSfi, liposomal curcumin, OYA-1, oxypurinol, mosedipimod, PUL-042, naltrexone, metenkefalin, COVID-EIG, TNX-1800, ATR-002, 177Lu-EC-Amifostine, 99mTc-EC-Amifostine, apabetalone, STI-6991, STI-4398, antroquinonol, ZIP-1642, DPX-COVID-19, belapectin, GX-19, AdCOVID, siltuximab, plitidepsin, C-21, meplazumab, pathogen-specific aAPC, LV-SMENP-DC, ARMS-I, rhu-pGSN, PRTX-007, CK-0802, namilumab, upamostat, NI-007, COVID-HIG, CYNK-001, Nafamostat, brilacidin, mavrilimumab, IPT-001, PittCoVacc, allo-APZ2-Covid19, ENU-200, VIR-7832, VIR-7831, pritumumab, Ampion, TZLS-501, sodium pyruvate, silmitasertib, CoroFlu, BDB-1, AT-001, BLD-2660, 20-hydroxyecdysone, IFX-1, elsulfavirine, emapalumab, CEL-1000, trabedersen, VBI-2901, ASC-09, TJM-2, RPH-104, tranexamic acid, WP-1122, olokizumab, APN-01, danoprevir, piclidenoson, FW-1022, CORAVAX, Lamellasome COVID-19, COVID-19 WG-03, EIDD-2801, AVM-0703, DC-661, acalabrutinib, bitespiramycin, Allocetra, tradipitant, bacTRL-Tri, Ad5-nCoV, EPV-CoV19, ADX-629, vazegepant, mercaptamine, sonlicromanol, aviptadil, fenretinide, IT-139, nitazoxanide, apabetalone, lucinactant, bacTRL-Spike, SAB-185, NVX-CoV2373, CM-4620, INO-4800, eicosapentaenoic acid, itanapraced, rintatolimod, XAV-19, niclosamide, ciclesonide, DAS181, ORBCEL-C, Metablok, dantrolene, CD24-IgFc, fadraciclib, gimsilumab, seliciclib, Cyto-MSC, ST-266, MRx-0004, ravulizumab, tafoxiparin, DAS-181, BMS-986253, cholecalciferol, nafamostat, ChAdOx1 nCoV-19, idronoxil, LY-3127804, ATYR-1923, VPM-1002, Mycobacterium w, lenzilumab, Polyoxidonium, conestat alfa, ubiquitin proteasome modulator, COVID-19 virus main protease Mpro inhibitor, mRNA-1273, clevudine, bucillamine, sodium meta-arsenite, vidofludimus, DARPin, COV-ENT-1, KTH-222, mefuparib, brensocatib, zanubrutinib, anakinra, selinexor, sarilumab, astodrimer, dapagliflozin propanediol, opaganib, BNT-162c2, BNT-162b2, BNT-162b1, BNT-162a1, ifenprodil, PIC1-01, 2X-121, zotatifin, aplidin, cloperastine, clemastine, dociparstat, avdoralimab, VIR-2703, ALN-COV, intravenous immunoglobulin (IVIg), apremilast, vicromax, baloxavir marboxil, emtricitabine, tenofovir, novaferon, secukinumab, valsartan, imatinib, omalizumab, leucine, sofosbuvir, alovudine, zidovudine, R-107, AB201, sargramostim, LYT-100, senicapoc, fluvoxamine, aspirin, losartan, ADX-1612, ADX-629, sirikumab, otilimab, STI-1499, TR-C19, ABX-464, interferon alpha2b, arbidol, 5309, vafidemstat, AT-527, ibudilast, auxora, bemcentinib, eculizumab, JS016, FSD-201, LY-CoV555, avifavir, OP-101, RLF-100, DMX-200, 47D11, remsima, TYR1923, dexamethasone, EDP-1815, PTC29, rabeximod, foralumab, budesonide, molnupiravir, ensovibep, dalcetrapib, FSD201, pralatrexate, proxalutamide, clofazimine and merimepodib.

25. The method of claim 1, wherein the patient receives standard of care co-treatment.

26. The method of claim 1, wherein the patient is also treated with corticosteroids.

27. The method of claim 1, wherein the patient is also treated with dexamethasone.

28. The method of claim 1, wherein the patient is also treated with remdesivir.

29. The method of claim 1, wherein the compound of formula 1, or a pharmaceutically-acceptable salt thereof, is administered to the patient at a dose of about 1 mg to about 10 mg.

30.-34. (canceled)

35. The method of claim 1, wherein the patient has acute lung injury associated with COVID-19.

36. The method of claim 1, wherein the patient has mild to moderate COVID-19.

37. The method of claim 1, wherein the patient has severe COVID-19.

38. The method of claim 1, wherein the patient is at high risk for progressing to severe COVID-19 and/or hospitalization.

39. The method of claim 1, wherein the patient suffers from hypertension and/or diabetes.

40.-66. (canceled)
